(19) United States
(12) Patent Application Publication
(10) Pub. No.: US 2026/0355328 A1
(43) Pub. Date: Nov. 12, 2026

(54) A complex comprising a cargo and a targeting moiety binding intelectin-1

(71) Applicant: Stichting Radboud universitair medisch centrum, Nijmegen (NL)

(72) Inventors: Johan van der Vlag, Arnhem (NL); Roland Erich Brock, Kalkar (DE); Jürgen Wilhelmus Carolus Dieker, Wijchen (NL); Alexander Henrik Baron van Asbeck, Nijmegen (NL)

(73) Assignee: Stichting Radboud universitair medisch centrum, Nijmegen (NL)

(21) Appl. No.: 19/114,806

(22) PCT Filed: Oct. 13, 2023
(86) PCT No.: PCT/EP2023/078533

§ 371 Date: Mar. 25, 2025

(30) Foreign Application Priority Data
Oct. 13, 2022 (EP) 22201291.6

(51) Int. Cl. A61K 47/62; A61K 47/54; A61K 47/60; A61P 13/12

(52) U.S. Cl. CPC A61K 47/62; A61K 47/549; A61K 47/60; A61P 13/12

(57) ABSTRACT

The invention relates to the field of drug delivery for inflammatory diseases, more specifically inflammatory kidney, eye, lung, and intestinal diseases, and kidney, eye, lung and intestinal diseases in general.

### FIELD OF THE INVENTION

The invention relates to the field of drug delivery for inflammatory diseases, more specifically inflammatory kidney, eye, lung, and intestinal diseases, and kidney, eye, lung and intestinal diseases in general.

### BACKGROUND OF THE INVENTION

As one class of kidney diseases, chronic kidney disease (CKD) affects an estimated 10% of the world population. CKD involves progressively destructive disease mechanisms, particularly in the glomeruli, the filtering units of the kidney.

Despite the growing prevalence and detrimental societal impact of CKD, so far, the treatment of patients is still limited to slowing down the progression of renal deterioration via the administration of systemically acting drugs. Next to interventions such as blood pressure lowering and dietary restriction, (often high initial doses of) anti-inflammatory drugs are applied, which include steroids (prednisone), mycophenolate mofetil, cyclophosphamide, azathioprine, and monoclonal antibodies such as Rituximab (anti-CD20) and Belimumab (anti-BLyS). Due to the general suppression of the immune system, these treatments lead to serious side effects such as fatigue, infections, infertility and cancer, which frequently are dose limiting. The use of (well) tolerated doses reduced the efficacy. As a consequence, under the current treatment paradigm, CKD ultimately leads to renal insufficiency and the need for regular dialysis and/or kidney transplantation with a considerable negative impact on quality of life and life expectancy. Renal targeting of (anti-inflammatory) drugs is considered a very promising way to circumvent these problems. However, targeting strategies that have been explored so far, including antibodies, sugars and liposomes, have not reached the clinic due to lack of glomerular targeting, too much off-targeting to other organs, and/or toxicity issues. Of note, most renal targeting strategies only reach tubular epithelial cells, whereas the glomerular cells are often the primary (start) site of kidney injury and permanent kidney function loss. Clearly, the current treatment with systemic application of anti-inflammatory/immunosuppressive drugs can only slow down deterioration of renal function and is associated with debilitating systemic side effects. These problems could be circumvented through the effective targeting of drugs to (glomerular) kidney cells. However, up to this day, no effective solution to this challenge has been presented.

### SUMMARY OF THE INVENTION

The invention relates to a complex comprising a cargo and a targeting moiety binding the intelectin-1 receptor (ITLN-1) for use as an agent to bind to, to target to, to purify, to induce uptake into cells or to transfect cells expressing ITLN-1 comprising administration of the complex to a subject, wherein the use preferably is therapeutic, diagnostic, prophylactic and/or theragnostic use, more preferably the use is for the treatment of a kidney disease.

The invention further relates to use of the complex according to the invention to bind, to target, to purify, to induce uptake, or to transfect cells expressing ITLN-1 comprising contacting the complex in vitro or ex vivo with the cells.

The invention further relates to an in vitro or ex vivo method to bind, to target, to purify, to induce (endosomal) uptake, or to transfect cells expressing ITLN-1 comprising contacting the complex according to the invention in vitro or ex vivo with the cells.

### DESCRIPTION OF THE INVENTION

All drug molecules act through interference with molecular processes inside the body. In some cases, activity throughout the whole body is desired and required as is the case for anti-coagulants and drugs that lower the blood pressure through influencing the physiology of capillary endothelial cells. In other cases, activity is restricted to specific cells and/or organs, because the targeted process is only present locally, despite a systemic distribution of the agent. An example would be substances interfering with the activity of neurotransmitters such as re-uptake inhibitors that exert their activity only on those cells that express the targeted protein. However, in most instances, drugs also act either on processes or on cells that do not play a role in the pathology for which the use of the drug is intended, commonly referred to as side effects. Side effects can arise for a number of reasons. First, next to the molecule that is the intended target for the relevant pathology, also other molecules are affected. The stereotypic example is acetyl-salicylic acid (Aspirin) which inhibits both cyclooxygenase 1 and 2 (COX1/2), whereas for the anti-inflammatory effect, primarily inhibition of COX2 is required. Therefore, so-called super aspirins only inhibiting COX2 were developed even though these later failed due to a more complex biology of COX2 (Colville-Nash and Gilroy Drug News Perspect. 2000; 13 (10): 587-97). Second, cells in tissues other than the one to be associated with the pathology are sensitive to the action of the drug. Here, the stereotypic example are anti-cancer drugs which frequently are directed at molecular processes associated with rapid, uncontrolled cell growth. As a consequence, also other rapidly dividing cells such as cells in the bone marrow, mucosal cells and cells in the hair follicles are affected. Third, metabolites that arise through metabolic activity of the body (e.g. detoxification processes in the liver) can by themselves have unwanted side effects (Tang and Lu Drug Metab Rev. 2010; 42 (2): 225-49).

A well-acknowledged solution, in particular for the second and third problem, is the targeted delivery of drugs to the site of action. As a consequence, such targeting changes the biodistribution (via uptake and/or retention). Here, the drug is formulated in such a way that the concentration of the drug at the desired therapeutic target site is increased in comparison to those cells/organs in which the action of the drug is not desired (otherwise side effects are induced as for anti-cancer drugs) or required (because the cells/organ are not associated with the pathology). The latter, even when not primarily associated with the avoidance of side effects, can be economically important. Oligonucleotide drugs such as small interfering RNAs (siRNAs), antisense oligonucleotides (ASO) and messenger RNA (mRNA) may not necessarily cause side effects when reaching cells other than the intended target cells. However, these drugs are expensive to manufacture and thus combination with targeting strategies makes treatments more affordable. Nevertheless, also for oligonucleotide drugs, numerous scenarios exist in which specific targeting is required so that critical side effects are avoided.

For targeting of drug formulations, two fundamental principles can be distinguished, which are passive and active targeting. Passive targeting is based on tissue characteristics such as the permeability of the vascular endothelium. The principle is also described as the enhanced permeation and retention (EPR) effect (Wang and Thanou Pharmacol Res. 2010; 62 (2): 90-9). The EPR effect is the concept underlying the targeting of nanoparticle drug formulations to solid tumours. Due to their rapid growth the vascular endothelium of solid tumours is less organized than the one of healthy tissue and thus leaky instead of tightly sealed. As a consequence, nanoparticles of a certain size can leave the bloodstream to enter the solid tumour but not the healthy tissue. Once inside the tumour, the particles are retained due to incomplete fluid removal by malformed lymph-vessels, and thus preferentially release the drug at the target site. The first clinically approved drug formulation that made use of this targeting principle is the liposomal formulation of the cancer drug doxorubicin, called Doxil (Sousa et al. Cancer Chemother Pharmacol. 2018; 82 (5): 741-55). A limitation of all passive targeting strategies is the structure of the vascular endothelium of organs of the so-called reticuloendothelial system (RES), of which the liver is the largest and most prominent organ. The organs of the RES filter particulate matter out of the blood and thus there are also large openings between the endothelial cells. The physiology of the RES therefore limits the effectiveness and specificity of passive targeting to other organs and tissues (Danhier J Control Release. 2016; 244 (Pt A): 108-21).

More specific and controllable targeting can be achieved through the so-called active targeting strategies (Yoo et al. Cancers (Basel) 2019; 11 (5): 640). For active targeting, the drug itself or the drug delivery vehicle (carrier in which the drug is incorporated such as liposomes) is equipped with molecular entities (targeting ligands) that lead to capture of the drug (formulation) at cells or structures of the extracellular matrix that present molecular structures that specifically interact with these molecular entities. Typically, these presented molecular structures are proteins expressed by the target cells, however, also sugars of glycoproteins and glycolipids, lipids of the plasma-membrane or proteins of the extracellular matrix located adjacent or upstream of the target cells can serve as target structures.

On one hand, targeting ligands can be derived from natural molecules. Examples that have been/are being explored in the area of drug targeting are folate which specifically interacts with folate receptors that are overexpressed on the surface of many cancer cells (Martin-Sabroso et al. Pharmaceutics. 2021; 14 (1): 14) and N-acetylgalactosamine (GalNAc) that specifically binds the asialoglycoprotein receptor on hepatocytes. Small interfering RNA (siRNA) coupled to GalNAc (Givosiran) has been approved for clinical use for example for the treatment of acute intermittent porphyria (Yu and Tu Pharmacol Ther. 2022; 230:107967).

On the other hand, targeting ligands can be generated through molecular engineering. The best-known example of engineered targeting ligands are antibodies which can be selected for virtually any cell surface structure. Antibody-drug conjugates are a rapidly growing field of drug formulations specifically for the treatment of cancers as they reduce/minimize the systemic exposure to the drug and thereby enable the use of highly effective cytotoxic agents (Beck et al. Nat Rev Drug Discov. 2017; 16 (5): 315-37). Since the selection and engineering of antibodies for use in humans is a laborious process and antibodies are also large proteins and expensive to produce, also small molecular weight protein scaffolds have been developed such as DARPins, anticalins and affibodies (Gebauer and Skerra Annu Rev Pharmacol Toxicol. 2020; 60:391-415). In contrast to antibodies, these proteins can also be expressed in bacteria, are very stable and due to their small size show a better penetration of tissues. Even smaller than engineered proteins are peptides which are also used in drug targeting as exemplified by peptides targeting the transferrin receptor to achieve transport across the blood-brain-barrier (Mojarad-Jabali et al. Int J Pharm. 2022; 613; 121395), however, peptides receive less attention in targeted drug delivery as they show rapid proteolytic degradation and rapid renal excretion in their unmodified form. Moreover, it is more difficult to generate high-affinity peptide ligands than high-affinity protein ligands.

Also, for active targeting strategies, several points require attention in order to afford highly effective and specific cellular targeting: Ideally, the targeted structure should only be present on the target cells. The abovementioned antibody-drug conjugates are directed towards receptors that are overexpressed on target cells, however, low expression levels on other cells can cause side effects (Ceci et al. Pharmacol Ther. 2022; 236:108106). Furthermore, targeted structures that are directly accessible from the blood stream can be reached easier than structures for which tissue penetration is required. As mentioned above, tissue penetration is compromised when the size of the targeted drug formulation exceeds the size of the vascular fenestrae, capillary junctions, pores in the ECM, or other physical structure separating the drug formulation from its target cell. In addition, whereas high affinity binding is an advantage for binding to target structures that are directly accessible from the blood stream, it may counteract penetration into tissues through a phenomenon that is known as the binding site barrier (Fujimori et al. J Nucl Med. 1990; 31 (7): 1191-8). For nanoparticle drug formulations that enter into tissues, it has been shown that implementation of active targeting does not necessarily enhance tissue enrichment as accumulation at the target site is dominated by passive targeting and the EPR effect (Kown et al. J Control Release 2012; 164 (2) 108-14), therefore active targeting of nano-formulations is preferentially directed at cells accessible from the blood stream as for example endothelia cells.

Finally, once binding has occurred, most targeted drug formulations require cellular uptake for their activity. Since the targeted formulation is directed at cell surface receptors, in nearly all cases, this uptake occurs by receptor-mediated endocytosis. For effective targeting and intracellular delivery, it is thus important that (i) a receptor is targeted that shows effective uptake and that (ii) the targeting ligand binds the receptor in a way that endocytosis takes place. Once inside the endosome, typically, the drug has to be released from the carrier. For antibody drug conjugates this release occurs through cleavage of linkers between antibody and drug that specifically react to the molecular environment in the endosomes or subsequent lysosomes (Panowski et al. MAbs 2014; 6 (1): 34-45). Drugs conjugated to antibody-drug conjugates are then typically hydrophobic enough to cross the endolysosomal membrane and enter the cytosol where they exert their activity. For oligonucleotides, endolysosomal release has to occur in a manner in which the carrier also facilitates transfer of the oligonucleotide through the endolysosomal membrane. Efficient endosomal release is as important for a carrier to show activity as the cellular uptake and targeting itself (Van Asbeck et al. ACS Nano. 2013; 7 (5): 3797-807).

Oligonucleotides (ON) are an important group of upcoming therapeutic molecules, promising both high efficacy and high specificity for a wide range of (previously undruggable) diseases (Smith et al. Annu Rev Pharmacol Toxicol. 2019; 59:605-630). The class of ON comprises of antisense oligonucleotides, small interfering RNAs (siRNAs), microRNAs, messenger RNA (mRNA), other small RNAs, and various forms of DNA. Due to the phosphate groups in their backbones, ON are highly negatively charged and the same holds true for most ON with backbone modifications, including those with phosphorothioates (Khvorova et al. Nat Biotechnol 2017; 35 (3): 238-248). ON almost invariably require uptake into the cytosol or nucleus of the cell to exert their biological activity, even if their transcriptional or translational products are extracellularly active. Furthermore, for most ON, cell-specific uptake is a pre-requisite for their biological activity because of the presence of corresponding cellular factors, such as a specific mRNA that is to be repressed by a given pharmaceutical siRNA, or specific localization in cells that should express an mRNA for therapeutic benefit in the body due to their physical location, translation capacity, exocytosis capacity, and/or involvement in specific bodily processes. Both the molecular weight and the negative charge of ON limit unassisted uptake by cells in general and also provide limited options for control over biodistribution in the absence of targeting ligands. Therefore, ON are a stereotypical class of drugs that benefit greatly from uptake enhancement and targeting (Hammond et al. EMBO Mol Med. 2021; 13 (4): e13243).

In general, there are two approaches to mediate targeting of ON, either through direct covalent conjugation of a targeting ligand or through packaging into (targeted) nanoparticles (Roberts et al. Nat Rev Drug Discov. 2020; 19 (10): 673-694). Direct conjugation is restricted to the short-length ON (antisense and siRNA), because for long-length ON, the size and charge-mediated inhibition of uptake cannot be (easily) overcome by the targeting ligand alone. Furthermore, if the targeting ligand carries a positive charge, then aggregation into nanoparticles via electrostatic interactions can occur, compromising a well-defined covalent conjugation. For mRNA, long-noncoding RNA, self-amplifying RNA and large (protein encoding) DNA, packaging into nanoparticles is often the only viable option, because of the need to avoid degradation of the ON in plasma, due to the presence of RNases and DNases, the low cytosolic uptake by the target cells and innate immune responses to the ON (Dowdy et al. Nat Biotechnol. 2017; 35 (3): 222; 229). Such nanoparticles can be formed by electrostatically complexing the negatively charged ON with the targeting ligand itself if sufficient cationic charge is available in the targeting agent. Alternatively, the ON can be encapsulated/complexed in/on a (ionizable or cationic) delivery vehicle, which can be further functionalized with (receptor-) targeting ligands.

The properties of a nanoparticle that are currently understood to be required for efficient encapsulation/binding of the ON and efficient uptake of a nanoparticle in a target cell (not being a phagocytotic immune cell), such as a diameter between 50-250 nm, an excess of (ionizable) cationic charge over negatively charged phosphates of ON, and membrane-interacting properties, overlap the properties of endogenous systems that remove nanoparticles from the bloodstream. The most relevant of these endogenous systems are the ApoE-mediated trafficking system that targets lipid-based nanoparticles to the liver, and the Reticulo-Endothelial System (RES) that actively captures large (>250 nm) and cationic nanoparticles via phagocytosis (Francia et al. Bioconjug Chem. 2020; 31 (9): 2046-2059). To achieve targeting towards other tissues for nanoparticles, targeting ligands need to have a high affinity towards a (surface) property of the target cell, such as a receptor protein, and a low affinity towards non-target cell-surfaces including low affinity to plasma proteins (most prominently IgG and ApoE).

As one class of kidney diseases, chronic kidney disease (CKD) affects an estimated 10% of the world population. CKD involves progressively destructive disease mechanisms in the glomeruli, the filtering units of the kidney. Glomeruli consist of a tuft of microcapillaries that are responsible for the charge and size-selective filtration of the blood through the glomerular filtration barrier (GFB). The GFB is a 3-layered filter that consists of fenestrated glomerular endothelial cells lining the glomerular microcapillaries, the glomerular basement membrane consisting of a mesh network of extracellular matrix proteins, and glomerular epithelial cells, also called podocytes with interdigitating foot processes. The healthy kidney contains about a million of these filtration units which are connected to the tubules where selective reabsorption (and secretion) of solutes and water from the pro-urine occurs. In general, the glomerulus and renal interstitium are considered the more relevant compartments for CKD, whereas the tubular compartment seems more relevant for acute kidney injury.

Since specific renal pathologies are associated with different renal compartments and different cell types, targeted drug delivery to the cell type involved in a specific pathology would be most beneficial. Current treatment is based on systemic interventions that slow down the deterioration of kidney function. Next to interventions such as the control of blood pressure (preferably lowering the blood pressure) and fluid balance and dietary restriction, often high initial doses of anti-inflammatory/immunosuppressive drugs are applied. The general suppression of the immune system, as a consequence of such treatment regimes, is associated with serious side effects such as fatigue, infections, infertility, and cancer, which frequently are dose limiting. Limitation of the dose reduces the efficacy. Therefore, ultimately, many patients will experience kidney failure, with dialysis and kidney transplantation as the only remaining treatment options. Part of the efficacy problem originates from the inability to reach a sufficient drug concentration in the kidney. The aforementioned problems associated with current treatments of kidney disease could be circumvented through the effective targeting of (anti-inflammatory) drugs to (glomerular) kidney cells. Such targeting strategies should increase the concentration of drug at the target site and simultaneously avoid systemic side effects through reduction of total applied dose.

Due to the severity of systemic side effects, there is an urgent search for renal targeting of drugs in renal pathologies and a variety of strategies have been explored in animals, primarily rodents. These strategies are comprised of antibodies directed against antigens on glomerular endothelial cells (ICAM-1, VCAM-1, E-selectin) or podocytes (Ig-receptor), sugar-linked nanoparticles (chitosan), peptides, polymers, and liposomes (van Asbeck et al. J Control Release. 2020; 328:762-775). Most renal targeting strategies explored so far, only reach tubular epithelial cells, whereas the glomerular cells are often the primary site and/or start site of kidney injury. To the best of the inventor's knowledge no targeting approach for drug delivery exists that can specifically target glomerular cells, which are the primary cells involved in damage of the kidney filter.

The abovementioned targeting technologies have shown limitations, which involve problems with specificity (for instance with respect to targeting to other (inflamed) endothelia, lack of uptake in glomerular cells (uptake occurs only in proximal tubules, which render these not applicable for drugs or nanoparticulate systems that cannot cross the glomerular filtration barrier), a high off-targeting ratio to liver or spleen, biodegradability issues (polymers) and accumulation of material that may aggravate inflammation (antibodies)). Importantly, all strategies have so far only been used in pre-clinical studies and have not progressed to the clinical stage.

While antibodies seem to be the logical choice for targeting of specific cell surface receptors, for glomerular targeting they are contra-indicated. Accumulation of antibodies on the glomerular filtration barrier can aggravate inflammatory reactions through direct interaction with molecules on endothelial cells, the GBM or the basal side of podocytes, or indirectly via charged-driven deposition of immunocomplexes on the GBM (Tecklenborg et al. Clin Exp Immunol. 2018; 192 (2): 142-150). Furthermore, the apical/luminal side of tubular epithelial cells cannot be reached by antibodies unless the GFB is damaged. Antibody fragments and related scaffolds such as nanobodies and DARPins provide an alternative of a sufficiently low molecular weight to cross the GFB but have not been explored in the context of kidney targeting (Deonarain et al. Drug Discov Today Technol. 2018; 30:47-53).

For polymer conjugates a further development into approved drugs has not taken place. Although chitosan is biologically safe and FDA approved for non-parenteral applications, so far, the main application has been in permeation enhancement across epithelial barriers as chitosan opens tight junctions (Amidi et al. Adv Drug Deliv Rev. 2010; 62 (1): 59-82; Ahmed et al. Drug Des Devel Ther. 2016; 10:483-507). A co-formulation with morphine as a nasal uptake enhancer has been in phase III clinical trial (Stoker et al. Pain Med. 2008; 9 (1): 3-12). As another example, challenges in the development of polymer-based drugs have been described for N-(2-Hydroxypropyl) methacrylamide (HPMA) conjugates. Despite a multitude of clinical studies, primarily in the area of cancer therapy (Duncan et al. Adv Drug Deliv Rev. 2009; 61 (13): 1131-48), no conjugate has been approved for the clinic, yet.

A further group of molecules for which accumulation in the kidney was described are so-called cell-penetrating peptides (CPPs). However, no information is available on the specific kidney structure (glomerulus versus tubules) reached by these peptides and in most cases also not on the biodistribution of such peptides. Most CPPs are cationic and/or hydrophobic and mediate the cellular uptake of themselves and/or molecules they are conjugated with. Conjugation can be either covalent or through non-covalent association such as charge-driven complexation of the cationic CPPs with negatively charged oligonucleotides. A key characteristic that distinguishes CPPs from other peptides used for cellular targeting is the absence of a specific receptor through which the cellular interaction is mediated. Instead, it is assumed that the interaction occurs either with the lipid bilayer of the plasma membrane or through charge- and hydrogen-bond driven association with sugars of the glycocalyx. While these interactions are based on types of interactions that are also found for receptor-ligand interactions, they lack structural (shape) complementarity, and thus also specificity, making them unlikely candidates as targeting agent.

Intelectins, such as intelectin-1 (ITLN-1), form a specific class of lectins that were originally discovered in intestinal cells. Both the amino acid sequences and the 3D structure of vertebrate intelectins are highly conserved (Yang et al. Scan J Immunol. 2020; e12882). Among the suggested roles for ITLN-1 is the clathrin-dependent and lactoferrin-mediated uptake of iron in intestinal cells, where the receptor is present in lipid rafts as a GPI-anchored protein (Suzuki et al. Biochem. 2001; 40 (51): 15771-15779; Wrackmeyer et al. Biochem. 2006; 45 (30): 9188-9197; Akiyama et al. J Biochem. 2013; 154 (5): 437-448).

In lung epithelial cells, ITLN-1 acts in a secreted form to assist in the phagocytic clearance of microorganisms by specifically binding microbial glycans (Tsuji et al. Glycobiol. 2009; 19 (5): 518-526; Gu et al. Am J Physiol. 2010. 298 (3): L290-296; Wesener et al. Nat Struct Mol Biol. 2015; 22 (8): 603-610). Designated as omentin-1, ITLN-1 is also secreted by adipocytes to modulate insulin sensitivity and is associated with metabolic diseases such as diabetes (Jaikanth et al. Exp Clin Endocrin Diabetes 2013; 121 (7): 377-383). In addition, ITLN-1 has been found overexpressed in multiple tumors (Zhou et al. PLOS ONE 2013; 8 (12): e81662; Dan et al. Oncotarget 2015; 6 (18): 16168-1682).

Several ligands for ITLN-1 have been associated with the abovementioned roles of the receptor. Regarding the detection of bacterial glycans, ITLN-1 specifically recognizes a terminal, acyclic 1,2-diol that is present on galactofuranose and other microbial saccharides (Wesener et al. Nat Struct Mol Biol. 2016; 22 (8): 603-610). Binding to bacterial glycans is coordinated by a bound calcium ion and is similar between human and mouse ITLN-1. Binding to a saccharide ligand may modulate binding to its protein ligand, that is lactoferrin and vice versa (Sharma et al. Int J Macromol. 2018; 108:1010-1016). ITLN-1 is one of the many lactoferrin-binding receptors, which also include CD14, LDL-related protein-1 (LRP-1), TLR-2 and 4, asialoglycoprotein receptor, and cytokine receptor 4 (CXCR4) (Kell et al. Front Immunol. 2020; 11:1221). Binding of lactoferrin by ITLN-1 has mostly been described for intestinal epithelial cells. Overexpression of ITLN-1 in these cells increases uptake of lactoferrin in endosomes (Akiyama et al. J Biochem. 2013; 154 (5): 437-48). The N-lobe domain of human lactoferrin (hLF peptide (hLF)) has been shown to be required for uptake in intestinal epithelial cells, whereas the C-lobe is involved in binding to bacteria (Suzuki et al. Biochem. 2008 47 (41): 10915-20). Lactoferricin (residues 17-41) and lactoferrampin (residues 268-284) are formed after proteolytic cleavage in nature and possess anti-microbial and cell-penetrating activities. Lactoferricin has been shown to bind to proteoglycans on endothelial and epithelial cells (Mader et al. Am J Pathol. 2006; 169 (5): 1753-1766; Andersen et al. Med Virol 2004; 262-271); however, binding to specific receptors, including ITLN-1, has not been demonstrated. In addition, the lactoferrin binding domain to an unidentified (lymphocyte) receptor has been described in loops 28-34 and 38-45 (Legrand et al. Biochem. 1992 31 (38): 9243-51), residues 4-90 (Rochard et al. FEBS Lett. 1989; 255 (1): 201-4) or residues 39-40 and 20-37 (Mazurier et al. Adv Exp Med Biol. 1994; 357:111-9). In contrast, binding activity of lactoferrin to an unidentified liver receptor was shown to be located in the C-lobe of lactoferrin (Sitaram et al. Protein Expr Purif. 1998; 14 (2): 229036; Sitaram et al. Biochem K. 1997; 323:815-22). Omentin-1 shares the same sequence with ITLN-1 but acts as an adipocyte-secreted cytokine that has been associated with multiple respiratory, neurological, metabolic, and vascular diseases (Zhou et al. Int J Mol Sci. 2018; 19 (1): 73; Niu et al. Front Cell Dev Biol. 2022; 9:784035; Watanabe et al. Compr Physiol. 2017; 7 (3): 765-81). So far, for omentin-1 only an association with integrin receptors on macrophages has been described, other binding partners are unknown (Lin et al. Cardiovasc Med. 2021; 8:757926). To date, the exact tissue distribution of ITLN-1 remains somewhat obscure. In mice, ITLN-1 expression has been observed in various tissues, including intestinal and lung epithelium, ovary, a small fraction of cells in the spleen, thymus and liver, and certain regions of the brain (Gu et al. Lung Cell Mol Physiol. 2010; 298 (3); Suzuki et al. Biometals 2004; 17 (3): 301-309). Some staining of the brush border of the proximal tubules in the kidney was also observed, while mRNA expression in the kidney was found to be extremely low. In humans, prominent mRNA expression has been observed in heart, intestine, and thymus, with no detectable expression in other organs (including the kidney) (Suzuki et al. Biochem. 2001; 40 (51): 15771-15779; Tsuji et al. J Biol Chem. 2001; 276 (26): 23456-23463), while protein expression was predominantly found in intestine and bladder epithelia, and weakly in some cells of heart and kidney (Washimi et al. PloS One 2012 7 (7): e39889). Another study found mRNA expression in intestine, heart, spleen, and ovary, but in contrast to the studies mentioned above, protein expression in these tissues was exclusively observed in endothelial cells, hence the name endothelial lectin (Lee et al. Glycobiol. 2001 11 (1): 65-73). One should note that in all these studies different antibodies were used, and no study demonstrated the localization of ITLN-1 in the glomeruli. Therefore, the inventors are the first to demonstrate convincingly the presence of the receptor that is ITLN-1 on the glomerular endothelial cells. Furthermore, the inventors observed that ITLN-1 expression is increased after pro-inflammatory activation of glomerular endothelial cells by TNFα, concomitant with an observed increase in uptake of the peptides derived from the N-lobe of human and mouse lactoferrin, making the receptor a novel candidate for active targeting of (anti-inflammatory) drugs to the kidney.

Active targeting of the glomerulus may be uniquely achieved with the peptides of the invention as injection of full-length radioactive-labeled lactoferrin protein was shown to result in distribution to many organs, which would be in line with the use of multiple receptors by the full-length protein, including ITLN-1 and LRP-1 (Huang et al. J Biomed Sci. 2007; 14 (1): 121-128).

As described above, several ligands have been described for ITLN-1. Currently, none of these ligands has been successfully applied to specifically target ITLN-1 expressing cells in drug delivery, meaning a pre-dominantly ITLN-1-mediated bio-distribution upon addition of an ITLN-1 selective compound has not yet been achieved. Human ITLN-1 specifically binds to bacterial glycans, while it does not bind any human glycan epitopes. However, using bacterial glycans includes a danger of unwanted immune activation via ITLN-1 or other pattern recognition receptors. Although full-length lactoferrin protein has been used for targeting (Singh et al. J Drug Target. 2016; 24 (3): 212-23; Sabra et al. Int J Biol Macromol. 2020; 164:1046-1060), it harbors multiple binding sites on different parts of the protein that bind a variety of receptors that include ITLN-1, LRP-1, CD14, TLR-2 and 4, asialoglycoprotein receptor and CXCR4, along with heparan sulfate proteoglycans. In particular, LRP-1 is highly expressed in different cell-types within multiple tissues, including liver, spleen and kidney. Therefore, full-length lactoferrin is unsuited for specific targeting of a particular receptor and thus unsuited to achieve bio-distribution pre-dominantly in a single organ/cell-type. More specifically, full-length lactoferrin is unsuited for kidney targeting.

Examples in the prior art showing targeting of non-kidney tissues by lactoferrin-drug conjugates/complexes and/or lactoferrin-containing nanoparticles may also suffer from a variety of issues reducing or precluding proper binding to ITLN1, including:
1. conjugation and/or (electrostatic) interaction of full-length or fragments of lactoferrin with specific types of cargo (e.g., gold nanoparticles) may obscure the ITLN-1 binding domains,
2. the (composite) targeting properties of certain cargo-lactoferrin combinations may favor bio-distribution to cells and tissues not expressing ITLN-1,
3. the conjugated or (electrostatically) bound cargo may prevent access to ITLN via sterical hindrance,
4. differences in receptor (ITLN-1) density and presentation on the cell, for example due to another genetic background or immunological status in the model used, which may influence the binding under biologically relevant conditions, and/or
5. differences in ligand (lactoferrin or lactoferrin-derived peptides) density and presentation on the nanoparticle surface, for example due to protein corona formation or different internal/surface structure, may influence the binding under biologically relevant conditions.

Moreover, full-length lactoferrin protein is known to polymerize, with tetramers being particularly dominant under physiological conditions (Mantel C, Miyazawa K, Broxmeyer H E. Advances in, Experimental Medicine and Biology. 1994. 357:121-32; Bagby G C, Bennett R M Blood 1982. Blood. 60 (1): 108-12). Finally, full-length lactoferrin is unsuited for RNA delivery, since it has pyrimidine-specific ribonuclease activity (McCormick J J, Larson L J, Rich M A. Nature 1974. 251 (5477): 737-40).

The inventors have established that peptides derived from the N-terminal domain of the human lactoferrin protein act as a targeting peptide to glomerular cells and in particular to glomerular endothelial cells and glomerular epithelial cells (podocytes). In contrast to earlier findings that peptides from this region act as cell-penetrating peptides (CPPs) (PCT/EP2006/010271), the inventors demonstrated that uptake in glomerular endothelial cells and podocytes was mediated through binding to ITLN-1. Receptor-mediated uptake is fundamentally different from uptake as a CPP. It is a key assumption in the field of CPPs that uptake occurs in a receptor independent manner. The inventors demonstrated that uptake in glomerular endothelial cells was not due to cell-penetrating activity of the peptide according to the invention, and largely independent of cell surface proteoglycans, which was previously described for other cell lines (FIG. 1) (Duchardt et al. 2009; J Biol Chem. 284:36099-36108).

Receptor binding was validated in various ways, including the reduction of uptake after downregulation of receptor expression using siRNA. Importantly, peptide variants that lack CPP activity only showed uptake in ITLN-1-positive cells. When glomerular endothelial cells were challenged with tumor necrosis factor alpha (TNF-alpha) to mimic an inflammatory condition, surprisingly, ITLN-1 was upregulated (FIG. 2B), which coincided with an increase in the uptake of the targeting peptide in inflamed cells (FIG. 1).

The inventors confirmed that ITLN-1 was expressed in the glomeruli of mouse and human kidneys (previously unknown). Intravenous injection of a fluorescently-labelled lactoferrin-derived targeting peptide in mice, in which glomerular inflammation was induced by LPS, showed a pronounced distribution in glomeruli, whereas this distribution was absent in non-challenged control mice (FIG. 4C). In addition, further targeting peptides were developed through removal or addition of naturally occurring amino acid residues from the N-terminus and/or C-terminus of the original lactoferrin-derived targeting peptide. These modifications further improved the kidney/liver ratio as well as the kidney/lung and kidney/spleen ratios, as an example of an overall improved bio-distribution to the kidney (i.e., accumulation in other organs was also reduced compared to accumulation in the kidney) (FIG. 5). In the development of targeting strategies in general, even if the target organ can be reached, accumulation in these (liver, spleen, lung) organs is a challenge that is difficult to overcome. No distribution to endothelial cells was observed other than glomerular endothelial cells. As a consequence, the activity for delivery in vivo is not to be attributed to the activity of the peptide as a CPP which should yield general uptake in endothelial cells that face the blood stream, but rather to a receptor-mediated activity that has not been recognized by the prior art. The variants of the peptide also comprise molecules that were not covered by the prior art. Importantly, upregulation of expression of ITLN-1 promotes a more efficient delivery in inflammatory situations, which opens the possibility to target inflamed tissues during glomerular diseases. It is even conceivable that the most inflamed parts of a target tissue receive the highest dose, providing a novel mechanism to fine-tune dose within an organ or tissue based on need.

Intravenous injection into mice revealed that the lactoferrin-derived peptide primarily distributed to the kidney (FIGS. 3 and 4), and that glomerular staining was more prominent in a mouse model for glomerular inflammation (FIG. 4). No distribution to endothelial cells was observed other than to glomerular endothelial cells. Consequently, the activity for delivery in vivo is not to be attributed to the activity of the peptide as a CPP which should yield general uptake in endothelial cells that face the blood stream, but to a receptor-mediated activity. Instead of acting as a CPP, the inventors demonstrated that uptake in glomerular endothelial cells and podocytes was mediated through binding to ITLN-1. Receptor-mediated uptake is fundamentally different from the receptor independent uptake of a CPP. Whereas all cells have a glycocalyx, the expression of a receptor very much depends on a specific cell type and the functional state of the cell (cell cycle, response to inflammation etc.). Receptor binding therefore creates a molecular basis for cell type-specific targeting whereas activity as a CPP does not. The conclusion on receptor dependence instead of CPP activity was based on the fact that the mouse homolog of the lactoferrin-derived peptide (mLF), was taken up by glomerular endothelial cells with the same activity as hLF (FIG. 1A and C), whereas on Hela cells, that are a frequently used cell line for testing the activity of CPPs, this peptide hardly showed any uptake (FIG. 1B). Secondly, treatment of glomerular endothelial cells with tumour necrosis factor α (TNFα) that mimics an inflammatory environment increased the expression of the receptor (FIG. 2B) and this increased expression was accompanied by an increased uptake of the peptide (FIG. 1E). Thirdly, both receptor and peptide were taken up by clathrin-mediated endocytosis as shown by colocalization with the protein transferrin which is a well-established marker for clathrin-mediated endocytosis (FIG. 2C and D). Finally, suppression of ITLN-1 expression by siRNA resulted in a significant decrease of peptide uptake (FIG. 2E). It has been reported before, that ITLN-1 acts as a receptor for full-length lactoferrin. However, it was a surprising finding that uptake of these peptides is mediated by the receptor because of several reasons, including the fact that both linear peptides and circularized peptides (via the 2 cysteine residues present) bind the receptor outside the conformational constraints of the full-length lactoferrin protein, and the fact that the endocytosis of the receptor-peptide complex is substantially faster than the off-rate of the peptides of the invention from the receptor. The increased expression of ITLN-1 upon inflammation was a further surprising finding as little was known about the biology of this receptor and particularly not on glomerular endothelial cells. Taken together, it was also completely unknown that this receptor may be used for specific drug delivery to the kidney, and specifically in a situation of inflammation which is of particular interest and significance for the application of therapeutics for CKD affecting the glomeruli. Thus, the effective glomerular targeting during inflammation, when it is most needed for localized anti-inflammatory treatment, provides a unique feature not shown for any other glomerular targeting molecules, in so far those exist.

Alternative peptides sequences, created by truncating either or both of the N-terminus and C-terminus of the starting peptide, demonstrated an improved kidney/liver ratio while retaining the kidney targeting capacity (FIG. 5). In addition, the kidney/lung and kidney/spleen ratios also improved, demonstrating that several peptides substantially avoid all major off-target organs, while maintaining sufficient affinity for the receptor for targeting purposes. The variants of the peptide also comprise molecules that were not covered by the prior art. These variants involved a reduction in total positive charge that reduce liver targeting, through addition and/or removal of naturally occurring amino acid residues at the N-terminus and/or C-terminus of the original targeting peptide. In one or more peptide variants, such total positive charge was reduced by the introduction of negatively charged residues naturally occurring upstream or downstream in the polypeptide sequence of the wildtype human lactoferrin protein. In the patent application on the use of the hLF peptide as a cell-penetrating peptide (PCT/EP2006/010271), the importance of the disulphide bridge for activity was stressed. However, the data herein on the peptide variants shows that peptides without the disulphide bridge demonstrate an even more favourable kidney-to-liver ratio. Surprisingly, complexation of the peptides with mRNA also demonstrated an enhanced kidney targeting (FIG. 3A/B).

Based on the previous findings it was fully unexpected that the uptake by glomerular cells in vitro and in vivo occurred via a specific receptor and was not merely due to the CPP activity of these peptides. Furthermore, these alternative peptides also demonstrated that positively charged residues in the peptide, which are typically involved in the uptake of CPPs, were less important and even the source of off-targeting and thus distinguish these peptides from the prior art of use as a CPP. Importantly, upregulation of expression of ITLN-1 promotes a more efficient delivery in inflammatory situations, which opens the possibility to target inflamed tissues during glomerular diseases.

The inventors' experiments revealed that glomerular endothelial cells express ITLN-1 (FIG. 2A). The localization observed for ITLN-1 was at least partially on the plasma membrane of glomerular endothelial cells, however, since ITLN-1 is a GPI-anchored protein the question remains whether uptake is a consequence of constitutive turnover of the protein or whether binding of the hLF peptide enhances internalization. Receptor binding was validated in several ways, including the reduction of uptake after downregulation of receptor expression using siRNA (FIG. 2E). Importantly, peptide variants that lack CPP activity only showed uptake in ITLN-1-positive cells. When glomerular endothelial cells were challenged with TNFα to mimic an inflammatory condition, surprisingly, ITLN-1 was upregulated, which coincided with an increase in the uptake of the targeting peptide in inflamed cells. The inventors demonstrated the presence of ITLN-1 in glomeruli of non-diseased mice and mice with glomerular inflammation (FIG. 2F). This showed that ITLN-1 was expressed in glomerular cells and small vessels in the mouse kidney. Intravenous injection of a fluorescently-labelled targeting peptide in mice, in which glomerular inflammation was induced by LPS (Lipopolysaccharide), showed a pronounced distribution in glomeruli, whereas this distribution was absent in non-challenged control mice (FIG. 4C). Importantly, the presence of ITLN-1 on glomerular cells and the upregulation during (glomerular) inflammation was not previously known.

Expression of ITLN-1 has been described in multiple tissues and cell types, as described above. The inventors established that targeting ITLN-1 with lactoferrin-derived peptides according to the invention resulted in a surprising prominent targeting of the kidney, while other organs such as lungs, spleen and intestines contained only minor amounts of peptide (FIG. 3-4), contrary to what would be expected based on the expression pattern of ITLN-1. The experiments by the inventors also revealed that intravenous injection of hLF peptide variants, with a reduced amount of positively charged residues, further enhanced targeting to the kidney with minor enrichment in the liver, lung and spleen (FIG. 5). Together with the finding that ITLN-1 expression was not detected in the liver and spleen, this suggested that enrichment in the kidney was mainly due to ITLN-1 binding, while enrichment in the liver and spleen was due to charge-dependent endocytosis and not dependent on ITLN-1.

Intravenous injection of free peptide (i.e., a peptide of the invention not being part of a supramolecular nanoparticle structure, and not being conjugated to a cargo molecule that would restrict glomerular filtration based on size or charge) resulted in a localization in glomerular and tubular cells. The inventors demonstrated that coupling the hLF peptide, or its derivates, to PEG resulted in an exclusive localization in the glomeruli (FIG. 6). Within the GFB, the glomerular endothelial cells are fenestrated with holes of 50-100 nm, while the slit pore of podocyte foot processes has gaps of approximately 30 nm (Wartiovaara et al. J Clin Invest. 2004; 114 (10): 1475-83). Therefore, particles smaller than 30 nm will pass the filtration barrier and reach the proximal tubules, which are expert cells in reabsorption of different kinds of molecules. This cellular characteristic is corroborated by the fact that non-conjugated hLF peptide demonstrated tubular uptake, while no ITLN-1 was expressed in these cells. Therefore, conjugation of hLF to delivery vehicles larger than 30-50 nm results in an ITLN-1-dependent targeting that is exclusive to glomeruli via the blood.

Accordingly, in a first aspect there is provided a complex comprising a cargo and a targeting moiety binding the intelectin-1 (ITLN-1), for use as an agent to bind to, to target to, to purify, to induce (endocytotic, e.g., clathrin-mediated or macropinocytotic) uptake into cells or to transfect cells expressing ITLN-1 comprising administration of the complex to a subject. Preferably, the affinity of the binding of ITLN-1 by said moiety has an equilibrium dissociation constant (K_{D}) of greater than 10⁻⁴ to 10⁻⁶ (micromolar sensitivity), more preferably greater than 10⁻⁷ to 10⁻⁹ (nanomolar sensitivity) and even more preferably greater than 10⁻¹⁰ to 10⁻¹² (picomolar sensitivity).

In the embodiments herein, uptake into cells may occur by a process known to the person skilled in the art, such as endocytotic, e.g., clathrin-mediated or macropinocytotic uptake into cells. The complex is herein referred to as a complex according to the invention. The cargo is herein referred to as a cargo according to the invention. The targeting moiety binding ITLN-1 is herein referred to as a targeting moiety according to the invention. Intelectin-1 is interchangeably referred to as ITLN-1. ITLN-1 is known to the person skilled in the art as extensively set forward hereinabove. In the embodiments herein, ITLN-1 may be any lactoferrin binding intelectin-1, such as the human, bovine, murine, goat, sheep, or non-human primate lactoferrin binding intelectin-1. ITLN-1 may or may not be a variant lactoferrin receptor intelectin-1 that has its endogenous function. ITLN1 may be the human intelectin-1 or a variant thereof that has its endogenous function.

In the embodiments herein, the targeting moiety binding ITLN-1 may be any targeting moiety known to the person skilled in the art, that has the ability to bind ITLN-1.

In the embodiments herein, the term “cell” is interchangeably used with the term “target cell”.

In the embodiments herein, the term “to transfect cells” has its general meaning known to the person skilled in the art, i.e. the process of deliberately introducing a nucleic acid into a cell, preferably into the intracellular compartment wherein said nucleic acid would be active (e.g., the cytosol for an mRNA, the nucleus for most DNA vectors).

In the embodiments herein, the targeting moiety may be selected from the group consisting of a protein, a peptide, a peptidomimetic, a DNA, an RNA, a carbohydrate, a polymer, a heterocycle, and a lipid. A protein, a peptide, a peptidomimetic, a DNA, an RNA, a carbohydrate, a polymer, a heterocycle, and a lipid are known to the person skilled in the art and may be any protein, peptide, peptidomimetic, DNA, RNA, carbohydrate, polymer, heterocycle and lipid that has the ability to bind to ITLN-1.

In the embodiments herein, the targeting moiety may be a peptide. Such peptide may be any peptide that has the ability to bind to ITLN-1. The peptide is herein referred to as a peptide according to the invention.

In the embodiments herein, the peptide may be a fragment from the full-length lactoferrin protein, or a variant thereof that has the endogenous function of the lactoferrin protein, or a protein that is functionally and/or structurally related to lactoferrin with at least 1 region capable of binding to ITLN-1.

In the embodiments herein, the peptide may be a fragment from the lactoferrin protein or a variant thereof that has the endogenous function of the lactoferrin protein, wherein said peptide comprises or consist of at least 6, 7, 8, 9, 10, 11, 12, 13, 14, 15, 16, 17, 18, 19, 20, 21, 22, 23, 24, 25, 26, 27, 28, 29 or at least 30 consecutive amino acids of the lactoferrin protein or variant thereof.

In the embodiments herein, the lactoferrin protein may be a human, bovine, murine, goat, sheep, or a non-human primate lactoferrin or a variant thereof that has the endogenous function of the lactoferrin or a protein that is functionally and/or structurally related to lactoferrin with at least 1 region capable of binding to ITLN-1.

In the embodiments herein, the lactoferrin protein may be the human lactoferrin or a variant hereof that has the endogenous function of the human lactoferrin or a protein that is functionally and/or structurally related to human lactoferrin with at least 1 region capable of binding to ITLN-1.

In the embodiments herein, the human lactoferrin protein or a variant hereof that has the endogenous function of the human lactoferrin, may comprise or consist of an amino acid sequence that has at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or at least 99% sequence identity to the amino acid sequence as set forward in SEQ ID NO: 1. In the embodiments herein, the human lactoferrin protein may comprise or consist of the amino acid sequence as set forward in SEQ ID NO: 1.

In the embodiments herein, the bovine lactoferrin protein or a variant hereof that has the endogenous function of the bovine lactoferrin, may comprise or consist of an amino acid sequence that has at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or at least 99% sequence identity to the amino acid sequence as set forward in SEQ ID NO: 2. In the embodiments herein, the bovine lactoferrin protein may comprise or consist of the amino acid sequence as set forward in SEQ ID NO: 2.

In the embodiments herein, the peptide may have a length of about 6 amino acids to about 30 amino acids, thus the peptide may have a length of at least 6 amino acids to at most 30 amino acids. In the embodiments herein, the peptide may have a length of 6 amino acids to 30 amino acids, of 7 to 28, of 8 to 26, of 9 to 24, of 10 to 22, of 12 to 20, or of 14 to 18 amino acids. The peptide may have a length of 6, 7, 8, 9, 10, 11, 12, 13, 14, 15, 16, 17, 18, 19, 20, 21, 22, 23, 24, 25, 26, 27, 28, 29 or 30 amino acids. The peptide may or may not comprise non-naturally occurring amino acids while maintaining its targeting moiety abilities. If the peptide comprises a cystine amino acid, such cystine amino acid may be or may have been replaced by a non-naturally circulating amino acid-like moiety. The peptide may comprise one or more natural or non-natural unpaired or reduced cysteine residues that may be used for conjugation to other cysteines, maleimides, thiols, etc. The peptide may comprise a natural unpaired cysteine residue which can be coupled to a lipid-PEG containing a terminal maleimide. Such lipid-PEG conjugate can prior to or subsequent to coupling with the targeting moiety be incorporated into the complex. The peptide may comprise a plurality of natural or non-natural cysteines or non-natural variants thereof as for example homocysteine, with at least one of such cysteine residues available for conjugation to the cargo. The remaining cysteines may be utilized for modification and/or stabilization of the structure of the targeting moiety.

In the embodiments herein, the peptide may have a net charge of at most +7, more preferably +6, more preferably +5, even more preferably +4 or +3. In the embodiments herein, in the complex for use, the peptide may have a net charge of at most +3, such as +4, +5, +6 or +7 at physiological pH i.e. pH 7.4, or the peptide may have a net charge of at most +3, such as +4, +5, +6 or +7 at endosomal pH i.e. between pH 5.0 and 7.4.

In the embodiments herein, the peptide may comprise or consist of a peptide selected from the group consisting of:

SEQ ID NO: 3
(KCFQWQRNMRKVRGPPVSCIKR);
SEQ ID NO: 4
(KCLRWQNEMRKVGGPPLSCIKR);
SEQ ID NO: 5
(KCFQWQRNMRKVRGPPVSCIKRDS);
SEQ ID NO: 6
(CFQWQRNMRKVRGPPVSCIKR);
SEQ ID NO: 7
(KCFQWQRNMRKVRGPPVSC);
SEQ ID NO: 8
(RKVRGPPVSCIKR);
SEQ ID NO: 9
(RKVRGPPVSCIKRDS);
SEQ ID NO: 10
(RKVRGPP);
SEQ ID NO: 11
(KCFQWQRNMRKVRGPPVSCIKRD);
SEQ ID NO: 12
(KCFQWQRNMRKVRGPPVSCIK);
SEQ ID NO: 13
(KCFQWQRNMRKVRGPPVSCI);
SEQ ID NO: 14
(KCFQWQRNMRKVRGPPVSC);
SEQ ID NO: 15
(KCFQWQRNMRKVRGPPVS);
SEQ ID NO: 16
(KCFQWQRNMRKVRGPPV);
SEQ ID NO: 17
(KCFQWQRNMRKVRGPP);
SEQ ID NO: 18
(CFQWQRNMRKVRGPPVSCIKR);
SEQ ID NO: 19
(FQWQRNMRKVRGPPVSCIKR);
SEQ ID NO: 20
(QWQRNMRKVRGPPVSCIKR);
SEQ ID NO: 21
(WQRNMRKVRGPPVSCIKR);
SEQ ID NO: 22
(QRNMRKVRGPPVSCIKR);
SEQ ID NO: 23
(RNMRKVRGPPVSCIKR);
SEQ ID NO: 24
(NMRKVRGPPVSCIKR);
SEQ ID NO: 25
(MRKVRGPPVSCIKR);
SEQ ID NO: 26
(RKVRGPPVSCIKR);
SEQ ID NO: 27
(CFQWQRNMRKVRGPPVSCIKRD);
SEQ ID NO: 28
(FQWQRNMRKVRGPPVSCIKRD);
SEQ ID NO: 29
(QWQRNMRKVRGPPVSCIKRD);
SEQ ID NO: 30
(WQRNMRKVRGPPVSCIKRD);
SEQ ID NO: 31
(QRNMRKVRGPPVSCIKRD);
SEQ ID NO: 32
(RNMRKVRGPPVSCIKRD);
SEQ ID NO: 33
(NMRKVRGPPVSCIKRD);
SEQ ID NO: 34
(MRKVRGPPVSCIKRD);
SEQ ID NO: 35
(RKVRGPPVSCIKRD);
SEQ ID NO: 36
(FQWQRNMRKVRGPPVSCIKRDS);
SEQ ID NO: 37
(FQWQRNMRKVRGPPVSCIKRDS);
SEQ ID NO: 38
(QWQRNMRKVRGPPVSCIKRDS);
SEQ ID NO: 39
(WQRNMRKVRGPPVSCIKRDS);
SEQ ID NO: 40
(QRNMRKVRGPPVSCIKRDS);
SEQ ID NO: 41
(RNMRKVRGPPVSCIKRDS);
SEQ ID NO: 42
(NMRKVRGPPVSCIKRDS);
SEQ ID NO: 43
(MRKVRGPPVSCIKRDS);
SEQ ID NO: 44
(RKVRGPPVSCIKRDS);
SEQ ID NO: 45
(CFQWQRNMRKVRGPPVSC);
SEQ ID NO: 46
(FQWQRNMRKVRGPPVSC);
SEQ ID NO: 47
(QWQRNMRKVRGPPVSC);
SEQ ID NO: 48
(WQRNMRKVRGPPVSC);
SEQ ID NO: 49
(QRNMRKVRGPPVSC);
SEQ ID NO: 50
(RNMRKVRGPPVSC);
SEQ ID NO: 51
(NMRKVRGPPVSC);
SEQ ID NO: 52
(MRKVRGPPVSC);
SEQ ID NO: 53
(RKVRGPPVSC);
SEQ ID NO: 54
(CFQWQRNMRKVRGPPVS);
SEQ ID NO: 55
(FQWQRNMRKVRGPPVS);
SEQ ID NO: 56
(QWQRNMRKVRGPPVS);
SEQ ID NO: 57
(WQRNMRKVRGPPVS);
SEQ ID NO: 58
(QRNMRKVRGPPVS);
SEQ ID NO: 59
(RNMRKVRGPPVS);
SEQ ID NO: 60
(NMRKVRGPPVS);
SEQ ID NO: 61
(MRKVRGPPVS);
SEQ ID NO: 62
(RKVRGPPVS);
SEQ ID NO: 63
(CFQWQRNMRKVRGPPV);
SEQ ID NO: 64
(FQWQRNMRKVRGPPV);
SEQ ID NO: 65
(QWQRNMRKVRGPPV);
SEQ ID NO: 66
(WQRNMRKVRGPPV);
SEQ ID NO: 67
(QRNMRKVRGPPV);
SEQ ID NO: 68
(RNMRKVRGPPV);
SEQ ID NO: 69
(NMRKVRGPPV);
SEQ ID NO: 70
(MRKVRGPPV);
SEQ ID NO: 71
(RKVRGPPV);
SEQ ID NO: 72
(CFQWQRNMRKVRGPP);
SEQ ID NO: 73
(FQWQRNMRKVRGPP);
SEQ ID NO: 74
(QWQRNMRKVRGPP);
SEQ ID NO: 75
(WQRNMRKVRGPP);
SEQ ID NO: 76
(QRNMRKVRGPP);
SEQ ID NO: 77
(RNMRKVRGPP);
SEQ ID NO: 78
(NMRKVRGPP);
SEQ ID NO: 79
(MRKVRGPP);

SEQ ID NO: 80 (K C X3 X4 W Q X7 X8 M X10 X11 X12 X13 X14 P X16 X17 X18 C X20 X21 X22 X23), wherein X3 is F, R or Y, X4 is R or Q, X7 is any natural or unnatural amino acid, X8 is any natural or unnatural amino acid, X10 is R, K or ornithine, X11 is R, K or ornithine, X12 is V or L, X13 is R or G, X14 is G or A, X16 is P or S, X17 is V, L, I, X18 is S or T, C, X20 is I or V, X21 is R or K or ornithine, X22 is R or K or ornithine, and X23 is D, S, A or T;
SEQ ID NO: 81 (X1, X2, X3, X4, X5 P X7), wherein X1 is R or K or ornithine, X2 is R, K or ornithine, X3 is V or L, X4 is R or G, X5 is G or A, and X7 is P or S;
SEQ ID NO: 82 (M X2, X3, X4, X5, X6 P X8), wherein X² is R, K or ornithine, X3 is R, K or ornithine, X4 is V or L, X5 is R or G, X6 is G or A, and X8 is P or S;
SEQ ID NO: 83 (X1 X2 X3 X4 X5 P X7 X8 X9 C X11 X12 X13 X14), wherein X1 is R, K or ornithine, X2 is R, K or ornithine, X3 is V or L, X4 is R or G, X5 is G or A, X7 is P or S, X8 is V, L, or I, X9 is S or T, X11 is I or V, X12 is R, K or ornithine, X13 is R, K or ornithine, X14 is D, S, A or T;
SEQ ID NO: 84 (X1 X2 X3 X4 X5 P X7 X8 X9 C X11 X12 X13 X14 S), wherein X1 is R, K or ornithine, X2 is R, K or ornithine, X3 is V or L, X4 is R or G, X5 is G or A, X7 is P or S, X8 is V, L, or I, X9 is S or T, X11 is I or V, X12 is R, K or ornithine, X13 is R, K or ornithine, and X14 is D, S, A or T.

In the embodiments herein, the targeting moiety, preferably the peptide, may comprise a protective moiety and/or a label moiety. Such protective moiety and a label moiety may be any protective moiety and a label moiety known to the person skilled in the art.

In the embodiments herein, the targeting moiety, preferably the peptide, may be end-protected to increase the stability in biological solutions, towards the presence of degrading enzymes (such as exonucleases, exopeptidases and exoglycosidases), and towards the presence of chemical reactions affecting the termini of the targeting moiety. Such end-protecting modifications may include terminal D-aminoacids, acetylation of the N-terminus, amidation of the C-terminus, C-terminal modification with N-alkyl amides, aldehydes, esters, p-nitroanilide, 7-amino-4-methylcoumarin, poly-ethylene glycol (PEG), PAS (an oligopeptide, consisting out of proline, alanine and serine residues), poly(glycerol) (PG), polyvinylpyrrolidone (PVP), Poly(N (2 hydroxypropyl) methacrylamide) (PHPMA) and N-terminal modification with formyl, pyroglutamyl, fatty acids, urea, carbamate, sulfonamide, alkylamine, PEG, PAS, PG, PVP, or PHPMA. C-terminal amidation and N-terminal acetylation both remove charge from the ends of the peptide, which changes solubility, possibly bio-distribution and biological activity, as they do mimic native peptide bonds when the peptide is selected from the internal sequence of a protein, and are preferred embodiments of the invention.

Accordingly, in the embodiments herein, the protective moiety may be selected from the group consisting of terminal D-amino-acids, D-amino-acids, non-natural amino-acids, acetylation of the N-terminus, acylation of the N-terminus, amidation of the C-terminus, C-terminal modification or substitution with N-alkyl amides, aldehydes, esters, p-Nitroanilide, 7-amino-4-Methylcoumarin, a suitable fluorescent label and/or N-terminal modification or substitution with formyl, pyroglutamyl, fatty acids, urea, carbamate, sulfonamide or alkylamine, glycosylation, PEGylation, PASylation, a suitable fluorescent group, a chelator for an ion, preferably a radioactive isotope for an ion, such as diethylenetriaminepentaacetic acid (DTPA) or a comparable substance.

In the embodiments herein, the targeting moiety, preferably the peptide, may be stabilized in biological solutions such as by a D-amino-acid, a retro-inverso sequence, a modified backbone structure, such as peptidomimetics, peptoids, or pseudo-peptide bonds, and by cyclization such as by the formation of an intermolecular or intramolecular cystine bond.

In the embodiments herein, the label moiety may be any label moiety known to the person skilled in the art such as one or more selected from the group consisting of a fluorophore, a hapten (such as biotin, digoxigenin, etc.), a DNA-, PNA-, XNA-, or RNA-barcode, a stable isotope, click-chemistry substrates and a radioactive label.

In the embodiments herein, the cargo may be modified to increase stability in biological solutions towards e.g., the presence of degrading enzymes such as exonucleases, exopeptidases and exoglycosidases and towards the presence of chemical reactions affecting the termini of the targeting moiety. Such modifications of the cargo may include modifications of the nucleic acid (NA)-backbone (such as Phosphorothioate bonds, locked nucleic acids (LNA)), modifications of the NA base, modifications of the termini of the NA (such as capping), modifications of the sugar (such as 2′O-methylation of RNA), hybridization to other (more stable) NA, conjugation to a (stabilizing) polymer (such as poly(ethylene glycol) (PEG), polyaminoacids such as Poly(Proline-alanine-serine) (PAS), poly(glycerols) (PG), poly(N-vinylpyrrolidone) PVP, poly(carboxybetaine) (pCB), poly(sulfobetaine) (pSB), phosphobetaine-base polymers, carbohydrates (e.g. heparins), or poly(N-(2-Hydroxypropyl) methacrylamide) PHPMA), the use of end-protecting modifications of peptides and proteins such as terminal D-amino-acids, acetylation of the N-terminus, amidation of the C-terminus. C-terminal modification with N-alkyl amides, aldehydes, esters, p-Nitroanilide, 7-amino-4-Methylcoumarin, poly-ethylene glycol (PEG), PAS, PG, PVP, pCB, pSB, carbohydrates, or PHPMA, and/or N-terminal modification with formyl, pyroglutamyl, fatty acids, urea, carbamate, sulfonamide, alkylamine, PEG, PAS, PG, PVP, pCB, pSB, carbohydrates, or PHPMA.

Other modification may include that the cargo is shielded by low-affinity polymers or molecules such as PEG from non-specific interactions with off-target receptors and other bodily components, resulting in a net gain of targeting efficiency by the targeting moiety. The relative amount and length of such shielding polymer should be sufficient to reduce the available interaction surface to reduce the unwanted molecular interactions. In such cases, the shielding polymer may also partially or fully cover the targeting moiety, or reduce its accessibility. It is therefore, preferred to directly or indirectly conjugate the targeting moiety to a freely available end of the shielding moiety, rather than directly to the cargo. Such a freely available end may be modified with any suitable click-chemistry or conjugation chemistry, such as maleimide to couple (native) cysteine residues, vinyl reagents to tetrazine, methylcyclopropene reagents, TCO reagents to tetrazine, norbornene to tetrazine, dibenzocyclooctyne (DBCO) reagents to azide and alkyne reagents to azide. Said conjugations may be performed prior or after the coupling to or insertion in the cargo. When said conjugations are performed prior to the formation or association with the cargo, for example in the case of conjugation of a targeting peptide to a pegylated lipid forming a peptide-PEG-lipid conjugate, the overall characteristics must be compatible with the conditions used for formulation. For example, the peptide-PEG-lipid conjugate is ideally soluble at relevant concentrations in an organic solvent, such as ethanol, when used as part of a lipid nanoparticle composition.

In the embodiments herein, the cargo may be covalently or non-covalently bound to the targeting moiety. The cargo may be covalently bound to the targeting moiety by means of one or more naturally present functional groups, such as the amino-terminus, carboxy-terminus, side-chain amino group, side-chain carboxy group, or preferably cysteines, in said targeting moiety or by means of a terminal or side-chain functionality of a non-natural amino acid such as an azide or an aldehyde or an alkene or alkyne or isocyanate or isothiocyanate. Depending on the functionality in the cargo to form the covalent bond with the targeting moiety, the bond may be an amide bond, an ester bond, a thioester bond, a disulfide bond, a thioalkane formed through a Michael addition or another type of chemical functionality that results from conjugation of the binding partners.

In the embodiments herein, in the complex, a multitude of targeting moieties may covalently bound to the cargo, such as 2, 3, 4, 5, 6 or more targeting moieties or a dendrimeric structure of targeting moieties.

In the embodiments herein, the affinity of the complex to ITLN-1 and/or ITLN-1 expressing cells may be enhanced by the multivalent use of the targeting moiety. The affinity may be enhanced by at least 2-fold, 3-fold, 4-fold, 5-fold, 6-fold, 7-fold, 8-fold, 9-fold or by at least 1 log.

In the embodiments herein, the cargo may be any useful moiety for the purpose of use within a complex to bind to, to target to, to purify to, to induce uptake into cells or to transfect cells expressing ITLN-1. The cargo may be a nucleic acid (e.g. a small interfering RNA (siRNA), anti-sense oligonucleotide (AON or asRNA), microRNA (miRNA), RNA aptamers, messenger RNA, trans-amplifying RNA, self-amplifying RNA, long non-coding RNA, split-replicon RNA, DNA aptamer, plasmid DNA, viral DNA, viral RNA, doggy-bone vector, circular RNA), a peptide (e.g. hormone-mimicking peptides, protein-binding peptides, RNA or DNA binding peptides, cytokines, growth factors), polypeptide or protein (e.g. anti-inflammatory proteins, immunomodulatory proteins, pro-resolving proteins, proteins affecting the redox-status and anti-oxidant response of the cell, ligand-binding receptors, signaling proteins, transcription factors), a carbohydrate (e.g. monosaccharides, disaccharides, oligosaccharides, and polysaccharides), a lipid, a polymer, a small molecule, or combinations or a mixture thereof. The cargo may be a vehicle such as a particle, such as a micro-particle or a nano-particle, a liposome, a lipid nanoparticle, a polymer particle, a silica particle, a carbon-nanotube, a gold particle, or a lipid- or polymer-micelle.

In the embodiments herein, when the cargo is a vehicle, the cargo may comprise any useful compound. The particle may comprise a pharmaceutically acceptable compound such as a pharmaceutically active compound (e.g. an anti-inflammatory compound (e.g. non-steroidal anti-inflammatory drugs such as aspirin, ibuprofen, naproxen, celecoxib, diclofenac, indomethacin, oxaprozin and/or piroxicam, more preferably steroidal inflammatory drugs such as prednisone, cortisone and methylprednisone, or anti-rejection drugs such as tacrolimus, cyclosporine, mycophenolate mofetil, azathioprine, rapamycin, sirolimus), an anti-cancer agent (e.g. chemotherapy such as alkylating agents (examples include Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin), nitrosoureas (examples include carmustine, lomustine, streptozocin), antimetabolites (examples include Azacitidine, 5-fluorouracil (5-FU), 6-mercaptopurine (6-MP), Capecitabine, Cladribine, Clofarabine, Cytarabine (Ara-C), Decitabine, Floxuridine, Fludarabine, Gemcitabine, Hydroxyurea, Methotrexate, Nelarabine, Pemetrexed, Pentostatin, Pralatrexate, Thioguanine, Trifluridine/tipiracil combination), anti-tumor antibiotics (examples include the anthracyclines Daunorubicin, Doxorubicin (Adriamycin), Doxorubicin liposomal, Epirubicin, Idarubicin, Valrubicin, and the anti-tumor antibiotics Bleomycin, Dactinomycin, Mitomycin-C, Mitoxantrone), topoisomerase inhibitors Irinotecan, Irinotecan liposomal, Topotecan, Etoposide (VP-16), Mitoxantrone, Teniposide), mitotic inhibitors (examples include the Taxanes Cabazitaxel, Docetaxel, Nab-paclitaxel and Paclitaxel, and the Vinca alkaloids Vinblastine, Vincristine, Vincristine liposomal, Vinorelbine), and other chemotherapy drugs (examples include All-trans-retinoic acid, Arsenic trioxide, Asparaginase, Eribulin, Hydroxyurea, Ixabepilone, Mitotane, Omacetaxine, Pegaspargase, Procarbazine, Romidepsin, Vorinostat)), cytokine drugs (including cytokines (examples include (recombinant versions of) IL-1, IL-2, TNF-alpha, IL-6, IL-7, IL-10, IL-12, IL-17, IL-21, IL-22, IL-23, IFN-alpha, IFN-beta, IFN-gamma, IFN-lambda1, IFN-lambda2, IFN-lambda3, IFN-omega, IP-10, MIP-1alpha, TGF-beta (1-3)) and anti-cytokines (e.g. cytokine-binding antibodies, decoy receptors, IL-1R antagonist)), growth-factors (examples include bone morphogenetic proteins (BMPs), vascular endothelial growth factor (VEGF), Granulocyte-macrophage colony-stimulating factor (GM-CSF), epidermal growth factor (EGF), erythropoeitin (EPO), insulin-like growth factor (IGF), fiboblast growth factor (FGF), hepatocyte growth factor (HGF), platelet-derived growth factor (PDGF), transforming growth factor (TGF), thrombopoietin (TPO)), hormones (examples include aldosterone, vasopressin, adrenocorticotropic hormone (ACTH), luteinizing hormone (LH), follicle-stimulating hormone (FSH), oxytocin, prolactin, thyroid-stimulating hormone (TSH), renin, angiotensin, glucagon, insulin, estrogen, progesterone, parathyroid hormone (PTH), Thyroid hormone, Epinephrine, Norepinephrine, testosterone, melatonin, growth hormone releasing hormone (GHRH), Thyrotropic releasing hormone (TRH), Gonadotropic releasing hormone (GnRH), corticotropin releasing hormone (CRH), humoral factors), cardiavascular medication (e.g. anti-coagulants (examples include Apibaxan, Dabigatran, Edoxaban, Heaprin, Rivaroxaban and Warfarin), anti-platelet agents (examples include Aspirin, Clopidogrel, Dipyridamole, Prasugrel and Ticagrelor), Angiotensin-Converting Enzyme (ACE)-inhibitors (examples include Benazepril, Captopril, Enalapril, Fosinopril, Lisinopril, Moexipril, Perindopril, Quinapril, Ramipril and Trandolapril), Angiotensin Il receptor blockers (Azilsartan, Candesartan, Eprosartan, Irbesartan, Losasartan, Olmesartan, Telmisartan, and Valsartan), beta-adrenergic blockers (Acebutolol, Atenolol, Betaxolol, Bisoprolol, MEtoprolol, Nadolol, Propranolol, and Sotalol), Calcium channel blockers (examples include Amlodipine, Diltiazem, Felodipine, Nifedipine, Nimodipine, Nisoldipine, and Verapamil), cholesterol-lowering medication (examples include Statins Atorvastatin, Fluvastatin, Lovastatin, Pitavastatin, Pravastatin, Rosuvastatin and Simvastatin, Nitotinic acid Niacin, and cholesterol absorption inhibitor Ezetimibe), digitalis preparations (Digoxin), diuretics (examples include Acetazolamide, Amiloride, Bumetanide, Chlorothiazide, Chlorthalidone, Furosemide, Hydro-chlorothiazide, Indapamide, Metalozone, Spironolactone, and Torsemide), and vasodilators (Isosorbide dinitrate, Isosorbide mononitrate, Hydralazine, Nitroglycerin and Minoxidil)), intestinal medication (e.g. Proton pump inhibitors (examples include Omeprazole, Lansoprazole, Rabeprazole, Esomeprazole, and Pantoprazole), Histamine2 blockers (examples include Cimetidine, Ranitidine, Famotidine, and Nizatidine), Promotility agents and laxatives (Metoclopramide)), eye medication (e.g. ocular allergy medicines (examples include Ketorolac, Ketotifen, loteprednol, Bepotastine, Epinastine, Emedastine, Alcaftadine, Azelastine, Olopatadine, Nedocromil, lodoxamide, and Cromolyn), topical antibiotics (examples include Besifloxacin, Ciprofloxacin, Moxifloxacin, Ofloxacin, Gatfloxacin, Tobramycin, Gentamycin, Polymyxin D, Neomycin, Bacitracin, Azithromycin, and Erythomycin), lipid-based artificial tears (examples include castor oil, glycerol, and mineral oil), NSAIDS and corticosteroids, glaucoma drugs (examples include Levobunolol, Timolol, Betaxolol, Bimatoprost, Travoprost, Latanoprost, Tafluprost, Brimonidine, Brinzolamide, and Dorzolamide), and antiviral treatment (examples include Acyclovir, Valacyclovir, and Famciclovir)), lung medication (e.g. anti-asthmatics (examples include dyphylline, guaifenesin, Albuterol, Levalbuterol), anti-histamines (examples include Brompheniramine, Carbinoxamine, Chlorpheniramine, Clemastine, Diphenhydramine, Hydroxyzine, Tripolidine, Azelastine, Cetrizine, Desloratadine, Fexofenadine, Levocetirizine, Loratadine, Olopatadine), Antitussives (examples include Dextromethorphan and Benzonatate), bronchodilators (Ipratropium, Theophylline, Albuterol, Epinephrine, Levalbuterol, Arformoterol, Formoterol, Olodaterol, Terbutaline, Pirbuterol, Metaproterenol, Salmeterol, Isoproterenol, Indacaterol, Tiotropium, Umeclidinium, Aclidinium, Ipratropium, Revefenacin, Glycopyrrolate, Ipratropium, Theophylline, Aminopylline and Dyphylline), decongestants (examples include Levmetamfetamine, Naphazoline, Oxymetazoline, Phenylephrine, Propylhexedrine, Pseudoephedrine, and Xylometazoline), expectorants (Guaifenesin), leukotriene modifiers (examples include Montelukast, Zafirlukast, Zileutron), lung surfactants (examples include Beractant, Lucinactant, Calfactant and Poractant), mucolytics (acetylcysteine), anti-infectives (examples include Zanamivir, Ribavirin, Tobramycin, Pentamidine, and Colistimethate), inhaled corticosteroids (examples include Fluticasone, Budesone, Mometasone, Beclomethasone, and Ciclesonide), mast-cell stabilizers (examples include Cromolyn and Nedocormil), and phosphodiesterase-4 inhibitors (including Roflumilast)), anti-microbial medication (including antibiotics (e.g. aminoglycosides (examples including Amikacin, Gentamycin, Kanamycin, Neomycin, Netilmicin, Tobramycin, Paromonmycin, Streptomycin and Spectinomycin), Ansamycins (examples include Geldanamycin, Herbimycin, and Rifaximin), Carbacephem (for example Laracarbef), Carbapenems (examples include Ertapenem, Doripenem, Imipenem, and Meropenem), Cephalosporins (examples include Cefadroxil, Cefazolin, Cephradine, Cephapirin, Cephalothin, Cefalexin, Cefaclor, Cefoxitin, Cefotetan, Cefamandole, Cefmetazole, Cefonicid, Cefprozil, Cefuroxime, Cefixime, Cefdinir, Cefditoren, Cefoperazone, Cefotaxime, Cefpodoxime, Ceftazidime, Ceftibuten, Ceftizoxime, Maxalactam, Ceftriaxone, Cefepime, Ceftaroline fosamil, and Ceftobiprole), Glycopeptides (examples include Teicoplanin, Vancomycin, Telavancin, Dalbavancin, and Oritavancin), Lincosamides (Clindamycin and Lincomycin), Lipopeptide (for example Daptomycin), Macrolides (Azithromycin, Clarithromycin, Erythromycin, Roxithromycin, Telithromycin, Spiramycin, and Fidaxomicin), Monobactams (for example Aztreonam), Nitrofurans (for example Furazolidone and Nitrofurantoin), Oxazolidinones (examples include Linezolid, Posizolid, Radezolid, and Torezolid), Penicillins (examples include Amoxiciliin, Ampicillin, Azlocillin, Dicloxacillin, Flucloxacillin, Mezlocillin, Methicillin, Nafcillin, Oxacillin, Penicillin G, Penicillin V, Piperacillin, Temocillin, and Ticarcillin), Polypeptides (examples include Bacitracin, Cilistin and Polymyxin B), Quinolones (examples include Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin), Sulfonamides (examples include Mafenide, Sulfacetamide, Sulfadiazine, Sulfadimethoxine, Sulfamethizole, Sulfamethoxazole, Sulfanilimide, Sulfasalazine, Sulfisoxazole, and Sulfonamidochrysoidine), Tetracyclines (examples include Demeclocycline, Doxycycline, Metacycline, Minocycline, Oxytartacycline, and Tetracycline), mycobacterium-specific antiobiotics (examples include (Clofazimine, Dapsone, Capreomycin, Cycloserine, Ethambutol, Ethionamide, Isonioazid, Pyrazinamide, Rifampicin, Rifabutin, Rifapentine and Streptomycin)), anti-fungals (e.g. Polyene antimycotics (examples include Amphotericin B, Candicidin, Filipin, Hamycin, Natamycin, Nystatin, and Rimocidin), Azoles (examples include Imidazoles Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole and Tioconazole, Triazoles Albaconazole, Efinaconazole, Epoxiconazole, Fluconazole, Isavuconazole, Itraconazole, Posaconazole, Propiconazole, Ravuconazole, Terconazole, and Voriconazole, and Thiazole (for example Abafungin), Allylamines (examples include Butenafine, naftifine and terbinafine), Echinocandins (Anidulafungin, Caspofungin, and Micafungin), and Triterpenoids (for example Ibrexafungerp)), and anti-parasite drugs (e.g. the broad-spectrum Nitazoxanide, antiprotozoals (examples include Melarsoprol, Eflornithine, Metronidazole, Tinidazole, and Miltefosine), Antinematodes (examples include Mebendazole, Pyrantel pamoate, Thiabendazole, Diethylcarbamazine and Ivermectin), Anticestodes (examples include Niclosamide, Praziquantel, and Albendazole), antitrematodes (for example Praziquantel), and Antiamoebics (for example Rifampicin and Amphotericin B))), anti-diabetic medication (e.g. Insulin (analogues), Amylinomimetic drugs (for example Pramlintide), Alpha-glucosidase inhibitors (examples include Acarbose, and miglitol), Biguanides (e.g. metformin (analogues and combinations), Dopamine agonists (for example Bromocriptine), Dipeptidyl peptidase-4 (DDP-4) inhibitors (examples include Alogliptin, Linagliptin, Saxagliptin and Sitagliptin), Glucagon-like peptide-1 receptor agonists (examples include Albiglutide, Dualglutaide, Exenatide, Liraglutide, and Semaglutide), Meglitinides (examples include Nateglinide and Repaglinide), Sodium-glucose transporter (SGLT-2) inhibitors (examples include Dapagliflozine, Canagliflozine, Ertugliflozine and Empagliflozine), Sulfonylureas (examples include Glimepiride, Gliclazide, Glipizide, Glyburide, Chlorpropamide, Tolazamide and Tolbutamide), Thiazolidinediones (for example Rosiglitazone and Pioglitazone)), anti-viral medication (examples include Abacavir, Acyclovir, Adefovir, Amntadine, Ampligen, Amprenavir, Umifenovir, Atazanavir, Atripla, Oseltamivir, Zanamivir, Peramivir, Baloxavir, Bikctegravir, Emtricitabine, Tenofovir, Boceprevir, Bulevirtide, Cidofovir, Cobicistat, Daclatasvir, Darunavir, Delavirdine, Didanosine, Docosanol, Dolutegravir, Doravirine, Edoxudine, Efavirenz, Emtricitabine, Enfuvirtide, Ensivirtide, Ensitrelvir, Entecavir, Entravirine, Famciclovir, Fomivirsen, Fosamprenavir, Foscarnet, Ganiciclovir, Ibacitabine, Ibalizumab, Idoxuridine, Imiquimod, Insoine pranobex, Indinavir, Lamivudine, Letermovir, Lopinavir, Loviride, Maraviroc, Methisazone, Moroxydine, Nelfinavir, Nevirapine, Nitazoxanide, Norvir, Penciclovir, Pleconaril, Podophyllotoxin, Raltegravir, Remdesivir, Ribavirin, Rilpivirine, Rimantadine, Ritonavir, Saquinavir, Simeprevir, Sofosbuvir, Stavudine, Taribavirin, Telaprevir, Telbivudine, Tenofovir, Tiprenavir, Trifluridine, Trizivir, Tromantadine, Truvada, Umifenovir, Valaciclovir, Valganciclovir, Vicriviroc, Vidarabine, Zalcitabine, Zanamivir, and Zidovudine), or structural or functional analogues thereof) or a diagnostic compound.

In the embodiments herein, the peptide is covalently, electrostatically, hydrophobically, or via an intermediate molecule, or through a combination thereof linked to the cargo.

In the embodiments herein, the cell may be a eukaryotic cell. The eukaryotic cell may be a mammalian cell, such a human, bovine, murine, goat, sheep, or non-human primate cell. In the embodiments herein, the cell may be human cell. In the embodiments herein, the cell may be an ITLN-1-expressing kidney cell, such as a glomerular endothelial cell or a podocyte. In yet another embodiment, the cell may be an ITLN-1-expressing eye cell (such as corneal epithelial cell), ITLN-1-expressing intestinal cell, or ITLN-1-expressing lung cell.

In the embodiments herein, administration of the complex may be performed by any means known to the person skilled in the art. Since many of the applications will be pharmaceutical applications, the complex may be within a pharmaceutical composition comprising further pharmaceutical compounds such as a pharmaceutically active compound and a pharmaceutically acceptable excipient. Such compositions are also provided as part of the invention.

In the embodiments herein administration may be in the form of a spray (lung, skin, eye, nose, oro-pharyngeal, ear), a creme (skin), a lotion (skin), drug-depot/slow-release formulations, a shampoo (skin/hair), a pill (intestine), eye drops (eye), micro-needles (skin), a wash/instillation (intraperitoneal, permucosal, vaginal, oral), biomaterials for regenerative medicine (bone, cartilage, connective tissue, skin, vascular structures), a coating of (implantable) medical devices, or any other suitable form that may be administered to a subject in need of treatment. In the embodiments herein administration to the subject may be performed by injection, preferably by intravenous (IV) injection.

In the embodiments herein, administration of the complex to a subject may result in increased intracellular concentration of the cargo in the ITLN-1-expressing cell. In the embodiments herein, administration of the complex to a subject may result in biodistribution of the complex being predominantly to ITLN-1-expressing cells, such that the cargo accumulates at least 1.5-fold, at least 2-fold, at least 3-fold, at least 4-fold, at least 5-fold, at least 6-fold, at least 7-fold, at least 8-fold, at least 9-fold, preferably at least 10-fold more in the organ containing ITLN-1-expressing cells than in the off-target organs, wherein off-target organ refers to an organ not containing ITLN-1-expressing cells.

In the embodiments herein, administration of the complex to a subject results in biodistribution being predominantly kidney-specific, such that the cargo accumulates at least 1.5-fold, at least 2-fold, at least 3-fold, at least 4-fold, at least 5-fold, at least 6-fold, at least 7-fold, at least 8-fold, at least 9-fold, preferably at least 10-fold more in the kidney than in any of the off-target organs. In the embodiments herein, the use as an agent to bind to, to target to, to purify, to induce uptake into cells or to transfect cells expressing ITLN-1 comprising administration of the complex to a subject, may be therapeutic, diagnostic, prophylactic and/or theragnostic use.

In the embodiments herein, the use as an agent to bind to, to target to, to purify, to induce uptake into cells or to transfect cells expressing ITLN-1 comprising administration of the complex to a subject, may be for use in the treatment of a kidney disease, such as a chronic kidney disease. In the embodiments herein, the use as an agent to bind to, to target to, to purify, to induce uptake into cells or to transfect cells expressing ITLN-1 comprising administration of the complex to a subject, may be for use in the treatment of an inflammatory disease, such as an inflammatory kidney disease, such as an inflammatory indication causing chronic kidney disease.

In the embodiments herein, the use as an agent to bind to, to target to, to purify, to induce uptake into cells or to transfect cells expressing ITLN-1 comprising administration of the complex to a subject, may be for use in the treatment of a condition associated with upregulation, over-expression and/or increased availability of the ITLN1 receptor.

In the embodiments herein, the use as an agent to bind to, to target to, to purify, to induce uptake into cells or to transfect cells expressing ITLN-1 comprising administration of the complex to a subject, may be for use in the treatment of acute kidney injury.

In the embodiments herein, the use as an agent to bind to, to target to, to purify, to induce uptake into cells or to transfect cells expressing ITLN-1 comprising administration of the complex, may be for use as pre-treatment for kidney transplantation wherein the complex is administered corporal and/or extra-corporal as pre-treatment for kidney transplantation. Extra-corporal means that the complex is administered to the kidney to be transplanted after it has been removed from the donor. In the embodiments herein, administration of the complex to the subject may result in faster clearance of the cargo from the system (blood) compared to clearance of the cargo without targeting moiety. In the embodiments herein, the clearance of the cargo may be more than 95% complete in less than 1 month, preferably in less than 1 week, less than 1 day, less than 1 hour, less than 30 minutes, less than 20 minutes, less than 15 minutes, less than 10 minutes, less than 7 minutes, more preferably in less than 5 minutes.

The complex as defined in the embodiments herein may conveniently be used to bind to, to target to, to purify, to induce uptake into cells or to transfect cells expressing ITLN-1 comprising contacting the complex in vitro or ex vivo with the cells. The features of this second aspect are preferably those of the first aspect herein. In this aspect, the cells may or may not be part of an organ wherein the complex is administered extra-corporally.

In a third aspect, there is provided an in vitro or ex vivo method to bind to, to target to, to purify, to induce uptake into cells or to transfect cells expressing ITLN-1 comprising contacting the complex in vitro or ex vivo with the cells. The features of this second aspect are preferably those of the first aspect herein. In this aspect, the cells may or may not be part of an organ wherein the complex is administered extra-corporally.

### FIGURE LEGENDS

FIG. 1

Lactoferrin-derived peptides are preferentially taken up by mouse glomerular endothelial cells (mGEnC).

(A) Flow cytometric analysis of mGEnC incubated for 30 minutes with different concentrations of the respective fluorescein-labelled peptides, i.e., human lactoferrin-derived peptide (hLF), mouse lactoferrin-derived peptide (mLF, no CPP activity), and the known cell-penetrating peptides R9 and penetratin, demonstrating increased uptake of lactoferrin-derived peptides.

(B) Hela cells show uptake of hLF, R9 and penetratin, but not of mLF.

(C) Confocal microscopy of mGEnC after incubation with 5 μM of the respective peptide for 30 minutes showing localization in endosome-like structures.

(D) Digestion of heparan sulphate from the cell surface has a limited effect on the uptake of lactoferrin-derived peptides (5 μM). Percentage of uptake for heparan sulphate-cleaved compared to undigested cells is shown.

(E) Treatment of mGEnC with tumour necrosis factor α (TNFα) increases uptake of 5 μM hLF (30 minutes).

FIG. 2

Uptake of the glomerular targeting peptide occurs via receptor-mediated endocytosis involving ITLN-1 as the receptor.

(A) Non-permeabilized cells (flow cytometry) and permeabilized cells (confocal microscopy) were stained with antibodies against ITLN-1 and low-density lipoprotein receptor-related protein 1 (LRP-1), both described as potential receptors for lactoferrin, showing the presence of ITLN-1 in/on mGEnC

(B). Treatment of mGEnC with TNFα increases protein expression of ITLN-1.

(C) hLF colocalizes with rhodamine-labelled transferrin indicating uptake via clathrin-coated pits.

(D) ITLN-1 also colocalizes with transferrin.

(E) Downregulation of ITLN-1 in mGEnC using siRNA decreases uptake of lactoferrin-derived peptides but not of the CPPs R9 and penetratin, and the transferrin receptor ligand transferrin.

(F) Frozen mouse kidney sections of normal BALB/c and MRL/MpJ mice (background strain), and MRL/MpJ-Fas^{Ipr} mice that have developed lupus-associated glomerular inflammation, were stained for ITLN-1 demonstrating expression in glomeruli.

FIG. 3

Biodistribution of 111 In-DOTA-hLF in female and male mice after intravenous injection.

(A) The peptide as free peptide (dark grey male and female symbols) or as particle with mRNA (light grey male and female symbols) shows high concentrations in kidney and urine, somewhat less in liver and at least 10-fold less in any other organ. Incorporation of hLF into nanoparticles demonstrates an improved targeting to the kidney. Gender is indicated by male/female symbols

(B) Normal BALB/c mice were intravenously injected with 111 In-labelled hLF as naked peptide or complexed with mRNA into 80 nm-sized nanoparticles (14 mice/group). Radioactivity was determined in indicated organs showing an increased distribution ratio of kidney to liver, spleen and lung. Statistic tests and differences are indicated in the graph.

(C) Blood is drawn from mice at several time points after peptide or polyplex injection, and amount of radiolabeled peptide is analysed. Peptide blood concentration is plotted as percentage of injected dose and decreases over time, with a clearance that is faster than the theoretical glomerular filtration rate (GFR), suggesting active binding/uptake.

FIG. 4 hLF distributes to glomeruli in an LPS-induced inflammation mouse model.

(A) Analysis of extracted organs after intravenous injection of Cy5.5-hLF in mice treated with LPS (left) or untreated (right), reveals a predominant distribution in kidney and liver.

(B) Confocal microscopy of organ sections shows renal localization of Cy5.5-hLF in (proximal) tubules and in glomeruli only in LPS-treated mice. Glomeruli are indicated by arrows. Liver, lung and spleen show a much lower fluorescence.

(C) Quantification of confocal pictures from the indicated parts of the kidney and other organs.

FIG. 5

Variants of hLF show improved targeting to the kidney.

(A) The percentage of total absorbed peptide that was detected in the kidney was calculated and depicted as violin plot for each peptide variant (upper left). The peptide sequences with respective charge are indicated in Table 1, as SEQ ID NO 90-97. The percentage injected dose per gram tissue was depicted for the kidney (lower left), liver (lower right) and spleen (upper right).

(B) In addition, the kidney/liver ratio for ¹¹¹In-labelled hLF variants with alterations to both the N- and C-terminal side of the peptide was determined. Box plots of the variants correspond to the sequences listed in Table 1.

FIG. 6

Analysis of mouse kidney slices after intravenous injection of the hLF variant coupled to PEG into an LPS-induced C57BI/6 mouse. Note that due to quenching and coupling efficiency, signal is expected to be lower compared to injecting non-conjugated peptide. Cy5.5 signal is pseudo-coloured, glomeruli are encircled.

### DEFINITIONS

“Sequence identity” is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. “Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g., the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps). Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons. Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg, gln or glu; Met to leu or ile; Phe to met, leu or tyr; Ser to thr; Thr to ser; Trp to tyr; Tyr to trp or phe; and, Val to ile or leu.

A “nucleic acid molecule” or “polynucleotide” (the terms are used interchangeably herein) is represented by a nucleotide sequence. A “polypeptide” is represented by an amino acid sequence.

A “nucleic acid construct” is defined as a nucleic acid molecule which is isolated from a naturally occurring gene or which has been modified to contain segments of nucleic acids which are combined or juxtaposed in a manner which would not otherwise exist in nature. A nucleic acid molecule is represented by a nucleotide sequence. Optionally, a nucleotide sequence present in a nucleic acid construct is operably linked to one or more control sequences, which direct the production or expression of said peptide or polypeptide in a cell or in a subject.

“Operably linked” is defined herein as a configuration in which a control sequence is appropriately placed at a position relative to the nucleotide sequence coding for the polypeptide of the invention such that the control sequence directs the production/expression of the peptide or polypeptide of the invention in a cell and/or in a subject. “Operably linked” may also be used for defining a configuration in which a sequence is appropriately placed at a position relative to another sequence coding for a functional domain such that a chimeric polypeptide is encoded in a cell and/or in a subject.

“Expression” is construed as to include any step involved in the production of the peptide or polypeptide including, but not limited to, transcription, post-transcriptional modification, translation, post-translational modification and secretion.

A “control sequence” is defined herein to include all components which are necessary or advantageous for the expression of a polypeptide. At a minimum, the control sequences include a promoter and transcriptional and translational stop signals. Optionally, a promoter represented by a nucleotide sequence present in a nucleic acid construct is operably linked to another nucleotide sequence encoding a peptide or polypeptide as identified herein.

The term “transformation” refers to a permanent or transient genetic change induced in a cell following the incorporation of new DNA (i.e. DNA exogenous to the cell). When the cell is a bacterial cell, as is intended in the present invention, the term usually refers to an extrachromosomal, self-replicating vector which harbors a selectable antibiotic resistance.

An “expression vector” may be any vector which can be conveniently subjected to recombinant DNA procedures and can bring about the expression of a nucleotide sequence encoding a polypeptide of the invention in a cell and/or in a subject. As used herein, the term “promoter” refers to a nucleic acid fragment that functions to control the transcription of one or more genes or nucleic acids, located upstream with respect to the direction of transcription of the transcription initiation site of the gene. It is related to the binding site identified by the presence of a binding site for DNA-dependent RNA polymerase, transcription initiation sites, and any other DNA sequences, including, but not limited to, transcription factor binding sites, repressor and activator protein binding sites, and any other sequences of nucleotides known to one skilled in the art to act directly or indirectly to regulate the amount of transcription from the promoter. Within the context of the invention, a promoter preferably ends at nucleotide-1 of the transcription start site (TSS).

A “polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

Sequence identity is preferably determined over the entire length of the subject sequence.

The sequence information as provided herein should not be so narrowly construed as to require inclusion of erroneously identified bases. The skilled person is capable of identifying such erroneously identified bases and knows how to correct for such errors.

The term ‘Active pharmaceutical compound/ingredient’ (API) does for the purpose of this invention include (messenger) RNA, oligonucleotides and DNA, even when the proposed activity is borne by the protein, polypeptide or peptide produced by the RNA or DNA, or regulated thereby.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a nucleic acid molecule or a peptide or polypeptide of a nucleic acid construct or vector or cell as defined herein may comprise additional component(s) than the ones specifically identified; said additional component(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 10% of the value.

All patent and literature references cited in the present specification are hereby incorporated by reference in their entirety.

Unless otherwise indicated each embodiment as described herein may be combined with another embodiment as described herein.

### Further Embodiments

Further embodiments of the invention are listed here below.
1. A complex comprising a cargo and a targeting moiety binding intelectin-1 (ITLN-1), for use as an agent to bind to, to target to, to purify, to induce uptake into cells or to transfect cells expressing ITLN-1 comprising administration of the complex to a subject.
1a. The complex for use according to embodiment 1, wherein said targeting moiety:
   a. specifically binds intelectin-1 (ITLN-1); and/or
   b. has sufficient affinity for ITLN-1 for stable binding; and/or
   c. does not comprise domains that can bind to other cell-surface receptors under biologically relevant conditions; and/or
   d. is accessible for binding by ITLN-1; and/or
   e. is not a full-length lactoferrin protein.
2. The complex for use according to embodiment 1 or 1a, wherein the targeting moiety is selected from the group consisting of a protein, a peptide, a peptidomimetic, a DNA, an RNA, a carbohydrate, a polymer, a heterocycle, and a lipid.
3. The complex for use according to embodiment 2, wherein the targeting moiety is a peptide.
4. The complex for use according to embodiment 3, wherein the peptide comprises or consists of at least 6 consecutive amino acids of a lactoferrin protein, or a variant hereof, preferably at most 300 consecutive amino acids, more preferably at most 100 consecutive amino acids, even more preferably at most 50 consecutive amino acids, most preferably at most 30 consecutive amino acids, preferably only comprising the ITLN-1 binding domain, a combination of ITLN-1 binding domains, or composite ITLN-1 binding domains.
4a. The complex for use according to embodiment 4, wherein the peptide is not full-length and/or wild-type lactoferrin protein
5. The complex for use according to embodiment 4 or 4a, wherein the lactoferrin protein is a human, bovine, murine, goat, sheep, or non-human primate lactoferrin.
6. The complex for use according to embodiment 5, wherein the human lactoferrin protein comprises or consists of the amino acid sequence as set forward in SEQ ID NO: 1 and the bovine lactoferrin protein comprises or consists of the amino acid sequence as set forward in SEQ ID NO: 2.
7. The complex for use according to any of embodiments 3 to 6, wherein the peptide has a length of about 6 amino acids to about 30 amino acids, preferably at least 6 to at most 30 amino acids.
8. The complex for use according to any of embodiments 3 to 7, wherein the peptide has a net charge of at most +7.
9. The complex for use according to any of embodiments 3 to 8, wherein the peptide comprises or consists of a peptide selected from the group consisting of: SEQ ID NO: 3 to 84.
10. The complex for use according to any of the preceding embodiments, wherein the targeting moiety, preferably the peptide, comprises a protective moiety and/or a label moiety.
11 The complex for use according to any of the preceding embodiments, wherein the targeting moiety, preferably the peptide, is stabilized in biological solutions.
12. The complex for use according to embodiment 10 or 11, wherein the protective moiety is selected from the group consisting of terminal D-amino-acids, D-amino-acids, non-natural amino-acids, acetylation of the N-terminus, acylation of the N-terminus, amidation of the C-terminus, C-terminal modification or substitution with N-alkyl amides, aldehydes, esters, p-Nitroanilide, 7-amino-4-Methylcoumarin, a suitable fluorescent label and/or N-terminal modification or substitution with formyl, pyroglutamyl, fatty acids, urea, carbamate, sulfonamide or alkylamine, glycosylation, PEGylation, PASylation, a suitable fluorescent group, a chelator for an ion, preferably a radioactive isotope for an ion, such as diethylenetriaminepentaacetic acid (DTPA) or a comparable substance.
13. The complex for use according to embodiment 10, wherein the label moiety is selected from the group consisting of a fluorophore, a hapten, a DNA-, PNA-, XNA-, or RNA-barcode, a stable isotope, click-chemistry substrates and a radioactive label.
14. The complex for use according to any of the preceding embodiments, wherein the cargo is covalently or non-covalently bound to the targeting moiety.
15. The complex for use according to embodiment 14, wherein the cargo is covalently bound to the targeting moiety by means of one or more naturally present functional groups, such as the amino-terminus, carboxy-terminus, side-chain amino group, side-chain carboxy group, or preferably cysteines, in said targeting moiety.
16. The complex for use according to embodiment 14 or 15, wherein a multitude of targeting moieties is covalently bound to the cargo, such as by a dendrimeric structure of targeting moieties.
17. The complex for use according to any of the preceding embodiments, wherein the affinity of the complex to ITLN-1 is enhanced by the multi-valent use of the targeting moiety, preferably at least 2-fold enhanced, more preferably at least 3-fold enhanced.
17a. The complex for use according to embodiment 17, wherein the affinity of the complex to ITLN-1 is affinity to ITLN-1 expressing cells.
18. The complex for use according to any one of the preceding embodiments, wherein the cargo is a nucleic acid, a peptide, polypeptide or protein, a carbohydrate, a lipid, a polymer, a small molecule or a mixture thereof.
19. The complex for use according to any one of the preceding embodiments, wherein the cargo is a vehicle such as a particle, such as a micro-particle or a nano-particle, a liposome, a lipid nanoparticle, a polymer particle, a silica particle, a carbon-nanotube, a gold particle, or a lipid- or polymer-micelle.
20. The complex for use according to any one of the preceding embodiments, wherein the particle comprises a pharmaceutically acceptable compound such as a pharmaceutically active compound or a diagnostic compound.
21. The complex for use according to any one of the preceding embodiments, wherein the peptide is covalently, electrostatically, hydrophobically, or via an intermediate molecule, or through a combination thereof linked to the cargo.
22. The complex for use according to any one of the preceding embodiments, wherein the binding is to a cell, wherein the cell is an ITLN1-expressing kidney cell, such as a glomerular endothelial cell or a podocyte.
23. The complex for use according to any one of the preceding embodiments, wherein administration to the subject is performed by injection, preferably by intravenous (IV) injection.
24. The complex for use according to any one of the preceding embodiments, wherein administration of the complex to a subject results in increased intracellular concentration of the cargo in the ITLN1-expressing cell.
25. The complex for use according to embodiment 24, wherein administration of the complex to a subject results in biodistribution of the complex being predominantly to ITLN1-expressing cells, such that the cargo accumulates at least 1.5-fold, at least 2-fold, at least 3-fold, at least 4-fold, at least 5-fold, at least 6-fold, at least 7-fold, at least 8-fold, at least 9-fold, preferably at least 10-fold more in the organ containing ITLN1-expressing cells than in the off-target organs, wherein off-target organ refers to an organ not containing ITLN1-expressing cells.
26. The complex for use according to embodiment 24 or 25, wherein administration of the complex to a subject results in biodistribution being predominantly kidney-specific, such that the cargo accumulates at least 1.5-fold, at least 2-fold, at least 3-fold, at least 4-fold, at least 5-fold, at least 6-fold, at least 7-fold, at least 8-fold, at least 9-fold, preferably at least 10-fold more in the kidney than in any of the off-target organs.
27. The complex for use according to any one of the preceding embodiments, wherein the use is therapeutic, diagnostic, prophylactic and/or theragnostic use.
27a. The complex for use according to embodiment 27a, wherein theragnostic use is a combination of therapeutic and diagnostic use.
28. The complex for use according to any one of the preceding embodiments, for use in the treatment of a kidney disease.
28a. The complex for use according to embodiment 28, wherein the kidney disease is chronic kidney disease.
28b. The complex for use according to embodiment 28 or 28a, wherein the cargo is a steroid, an anti-inflammatory drug, an anti-fibrotic drug, a steroidal anti-inflammatory drug, a protein kinase inhibitor, a gene-correcting agent, or an oligonucleotide, or wherein the cargo is messenger RNA, circular RNA, trans-amplifying RNA, self-amplifying RNA, or DNA, wherein the cargo is preferably an anti-inflammatory drug or an anti-fibrotic drug, wherein the cargo is preferably a nucleic acid.
29. The complex for use according to any one of the preceding embodiments, for use in the treatment of an inflammatory disease, preferably an inflammatory (chronic) kidney disease.
30. The complex for use according to any one of the preceding embodiments, for use in the treatment of a condition associated with upregulation, over-expression and/or increased availability of ITLN-1.
30a. The complex for use according to embodiment 30, wherein the condition is inflammation, such as kidney inflammation or ulcerative colitis, or wherein the condition is cancer, such as prostate cancer.
31. The complex for use according to any one of the preceding embodiments, for use in the treatment of acute kidney injury.
32. The complex for use according to any one of the preceding embodiments, wherein the complex is administered corporal and/or extra-corporal as pre-treatment for kidney transplantation.
33. The complex for use according to any one of the preceding embodiments, wherein clearance of the cargo from the system is accelerated compared to the cargo without targeting moiety, wherein clearance from the system is preferably clearance from the blood circulatory system.
34. The complex for use according to embodiment 33, wherein clearance of the cargo is more than 95% complete in less than 1 month, preferably in less than 1 week, less than 1 day, less than 1 hour, less than 30 minutes, less than 20 minutes, less than 15 minutes, less than 10 minutes, less than 7 minutes, more preferably in less than 5 minutes.
35. Use of the complex as defined in any one of embodiments 1 to 32, to bind, to target, to purify, to induce uptake, or to transfect cells expressing ITLN-1 comprising contacting the complex in vitro or ex vivo with the cells.
35a. The use according to embodiment 35, wherein the contacting is under conditions that allow for receptor-specific binding and/or uptake, including a concentration of less than 5 μM, pH between 6.0-7.5, duration of contact of at least 2 minutes up to 72 h, preferably 2-20 minutes, and a temperature between 15-37 °C.
36. An in vitro or ex vivo method to bind, to target, to purify, to induce (endosomal) uptake, or to transfect cells expressing ITLN-1 comprising contacting the complex as defined in any of embodiments 1 to 22 in vitro or ex vivo with the cells.
36a. The method according to embodiment 36, wherein the contacting is under conditions that allow for receptor-specific binding and/or uptake, including a concentration of less than 5 μM, pH between 6.0-7.5, duration of contact of at least 2 minutes up to 72 h, preferably 2-20 minutes, and a temperature between 15-37 °C.

### EXAMPLES

The following examples are offered for illustrative purposes only, and are not intended to limit the scope of the present invention in any way.

### Example 1

This example illustrates the specific uptake of the human lactoferrin (hLF) peptide in glomerular endothelial cells that is independent of its activity as a cell-penetrating peptide (CPP) that was earlier described (PCT/EP2006/010271).

### Methods

Conditionally immortalized mouse glomerular endothelial cells (mGEnC) were cultured as previously described (Rops et al. Kidney Int. 2004; 66 (6): 2193-201). HeLa cells were employed as a frequently used model cell line and were cultured in DMEM/F12 medium containing 10% fetal calf serum (FCS). For studying uptake, cells were incubated in their respective medium with 10% FCS containing fluorescein-labelled peptides at the indicated concentrations for 20 minutes at 37 °C. In some cases, cells were activated for 16 hours with 10 ng/ml mouse TNFα (Sigma), or co-incubated with Alexa Fluor 633-labeled transferrin (Invitrogen Molecular Probes). Subsequently, cells were washed with PBS and analysed using confocal laser-scanning microscopy on a TCS SP5 confocal microscope (Leica Microsystems), or detached by trypsin/EDTA and analysed on a FACSCalibur Flow cytometer (BD Biosciences) by measuring 20,000 cells.

### Results

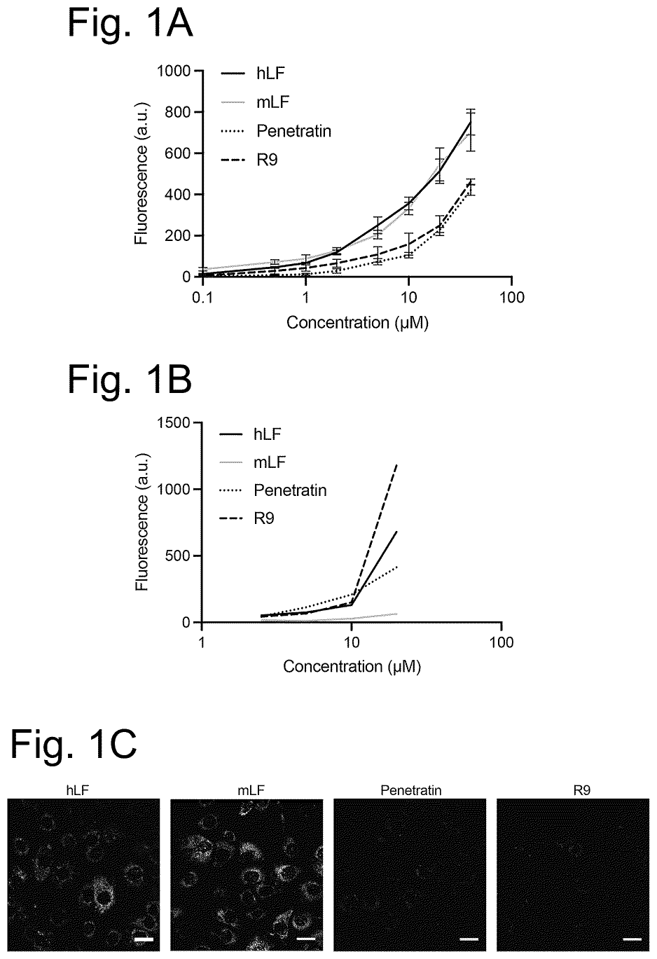

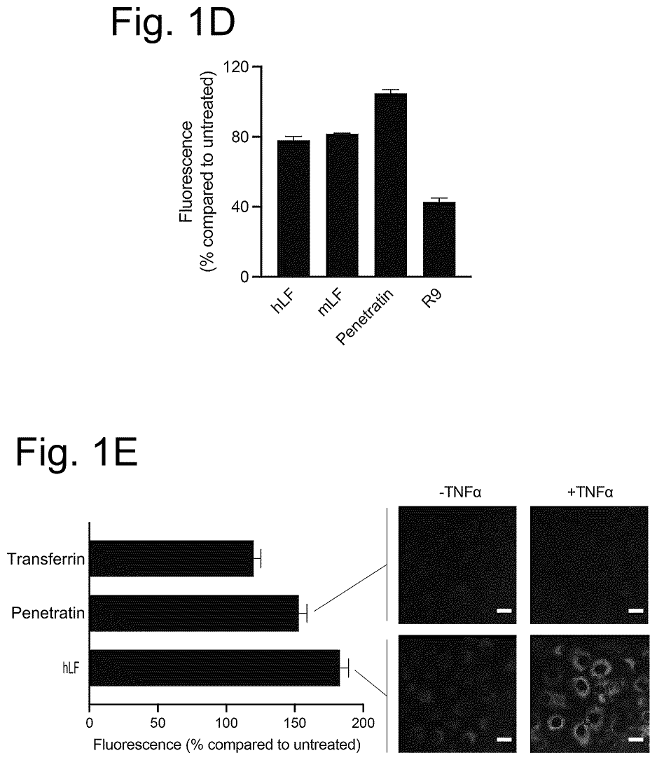

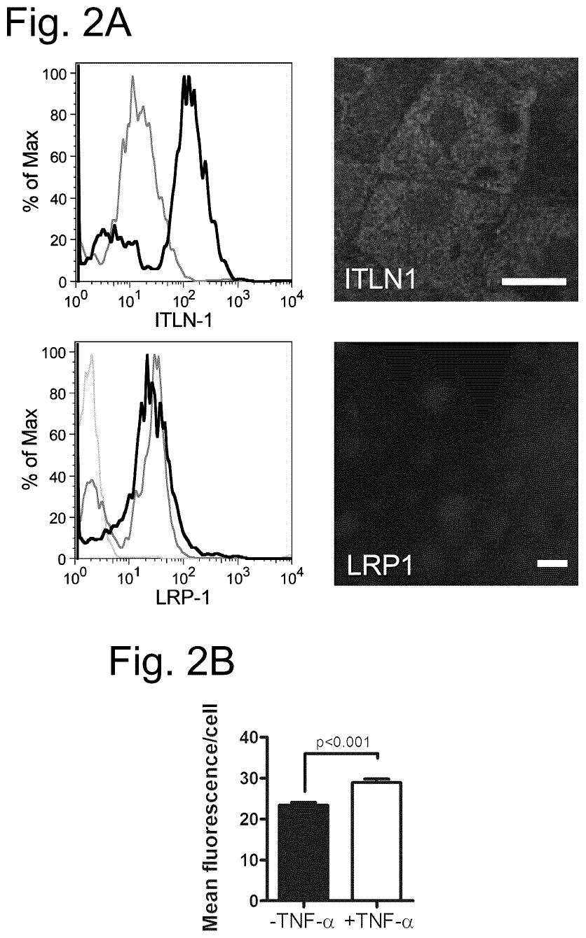

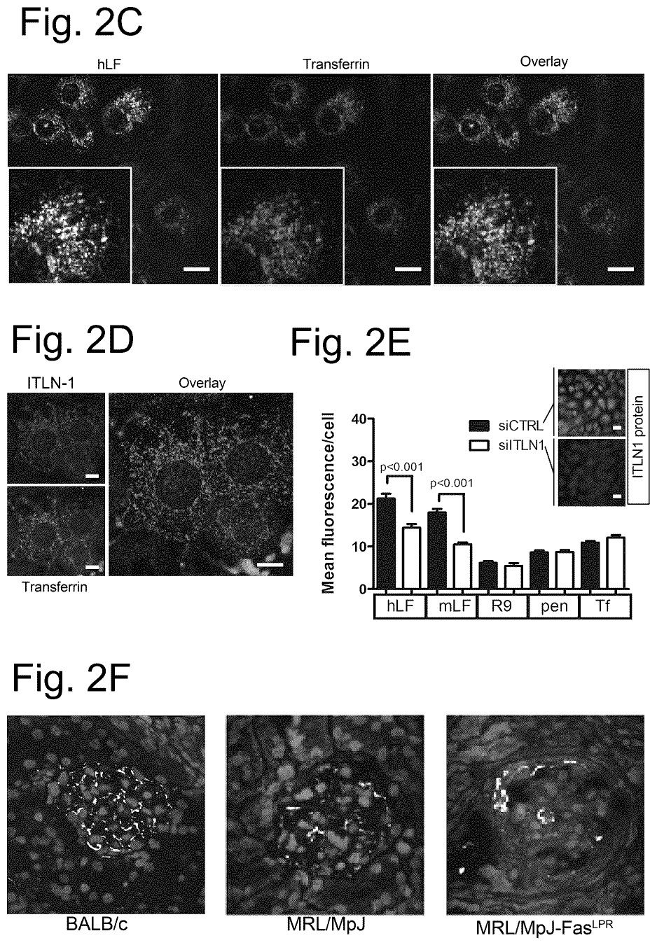

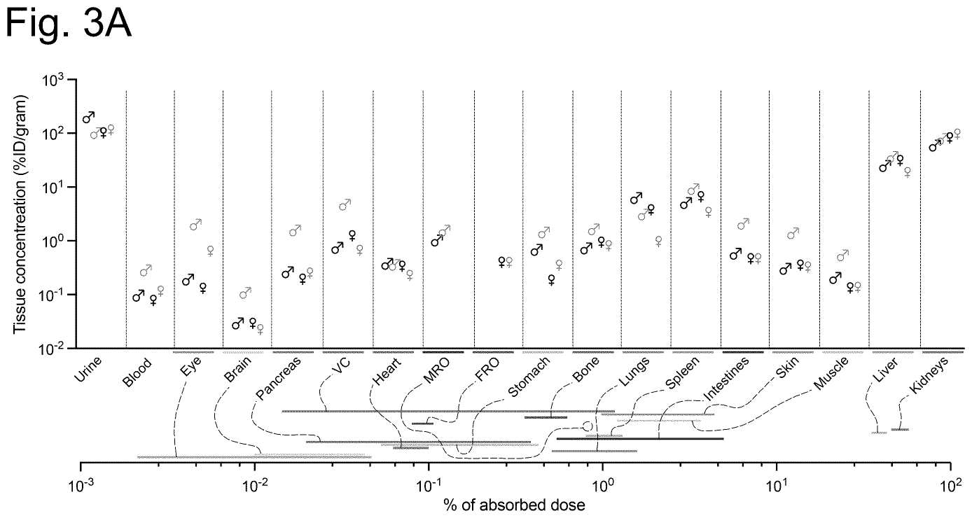

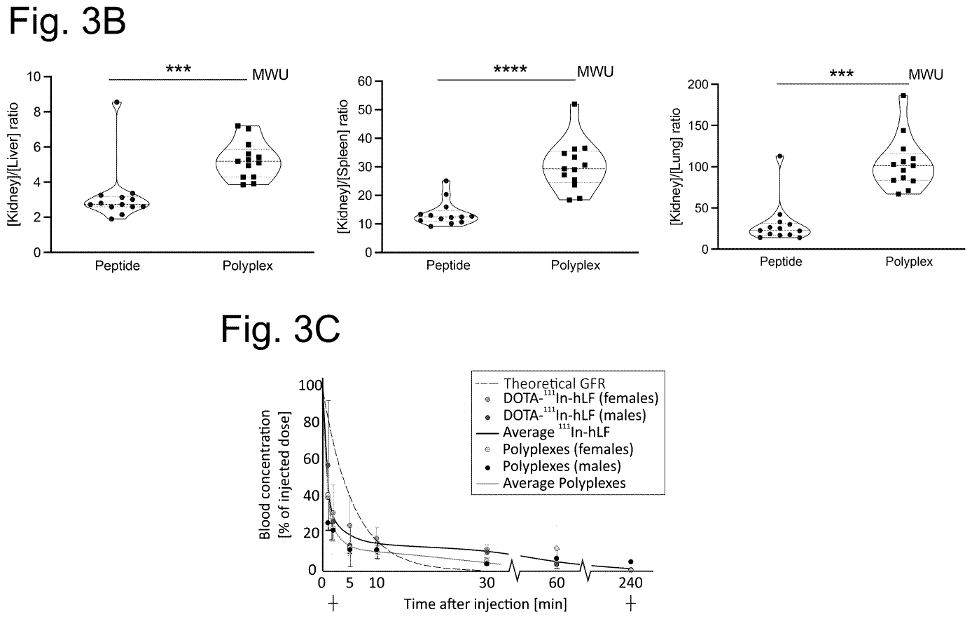

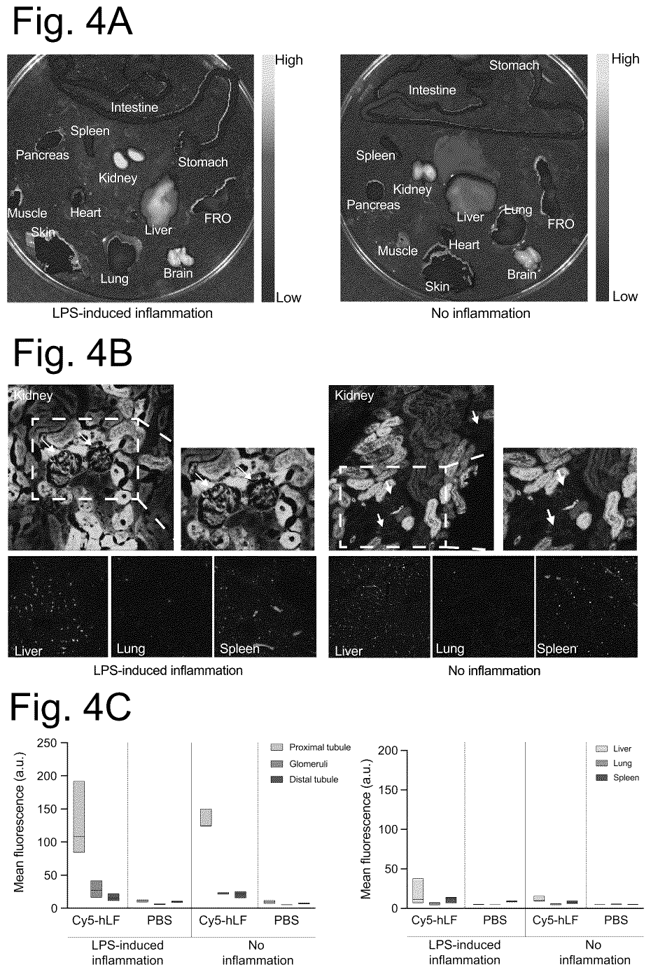

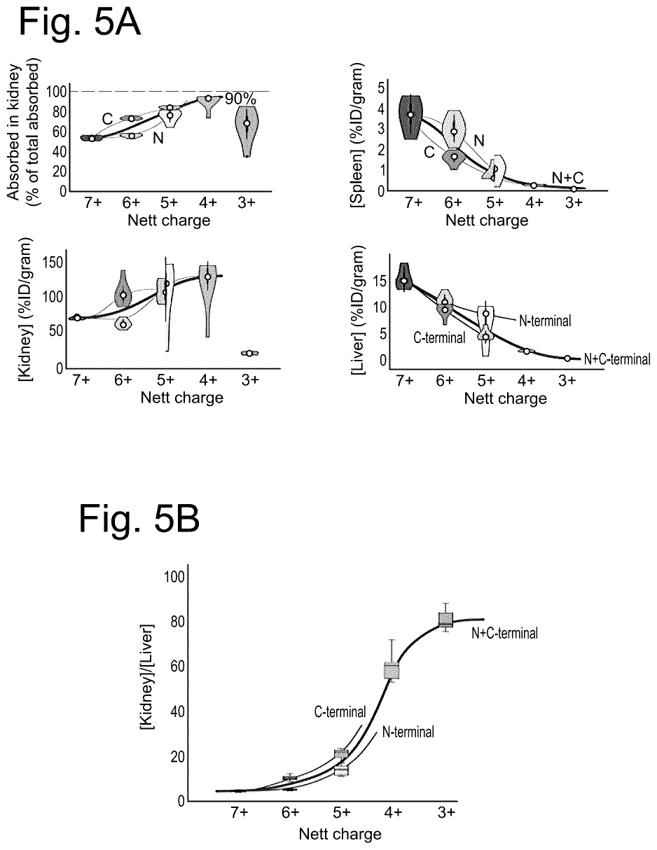

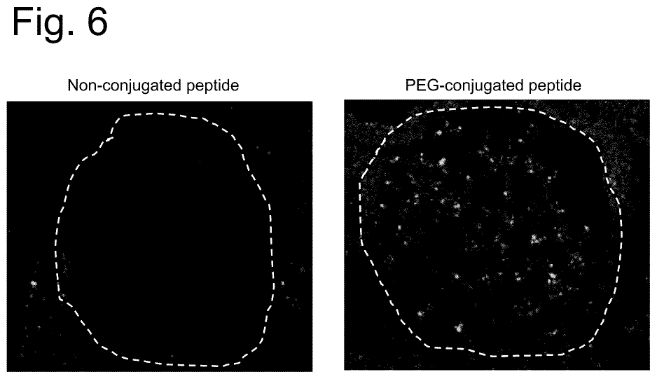

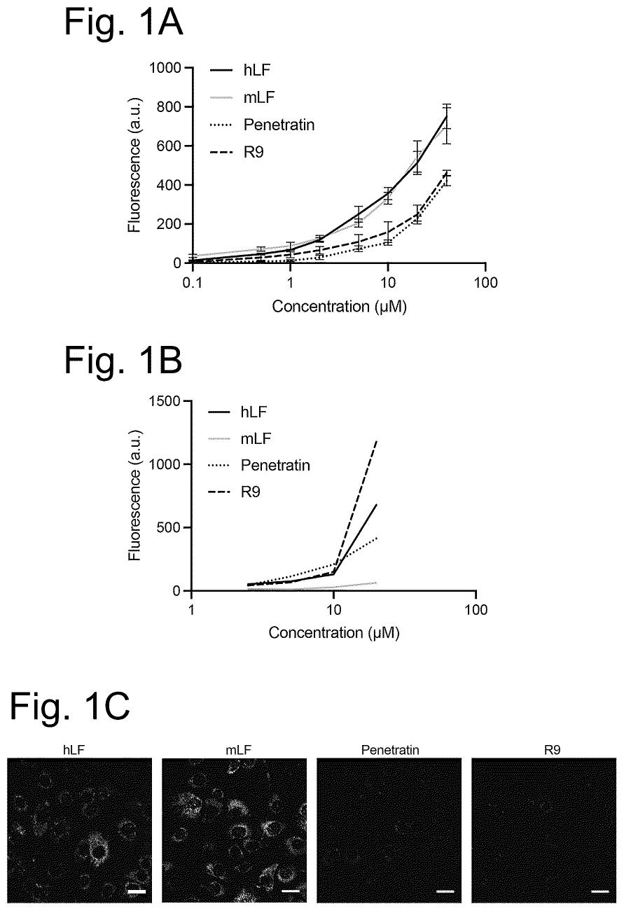

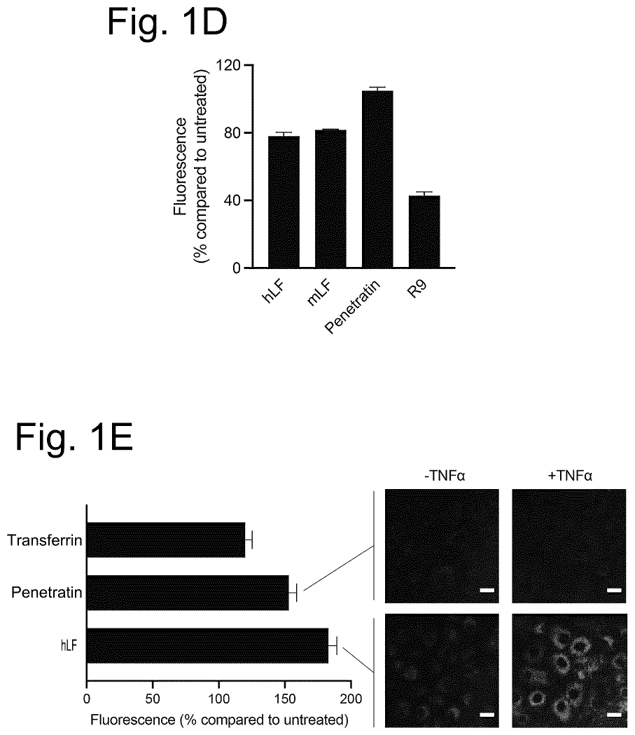

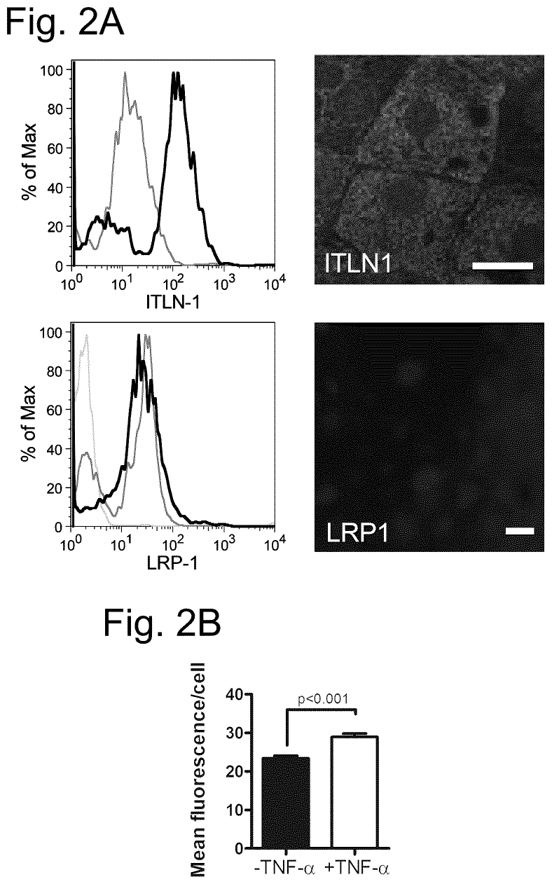

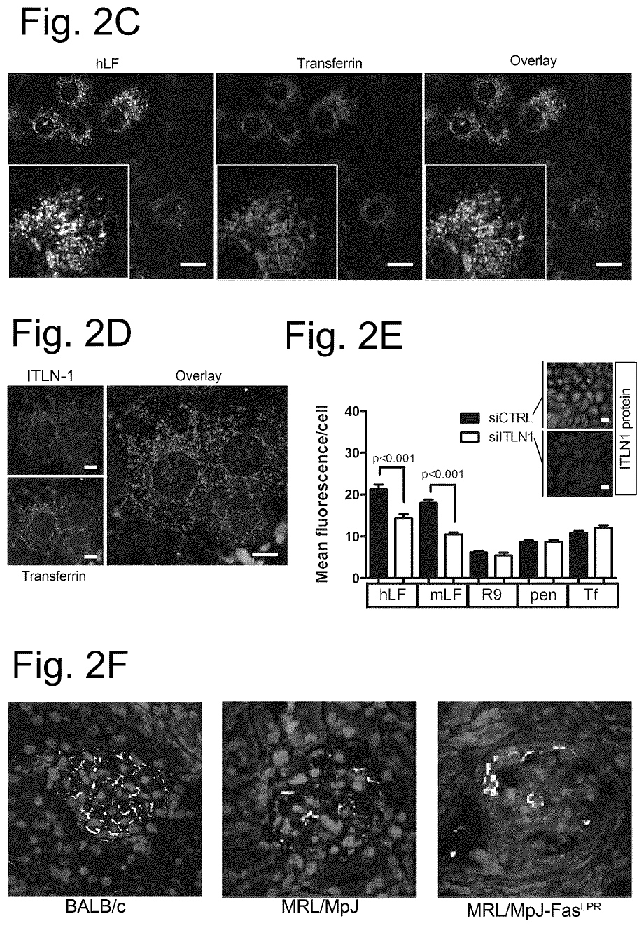

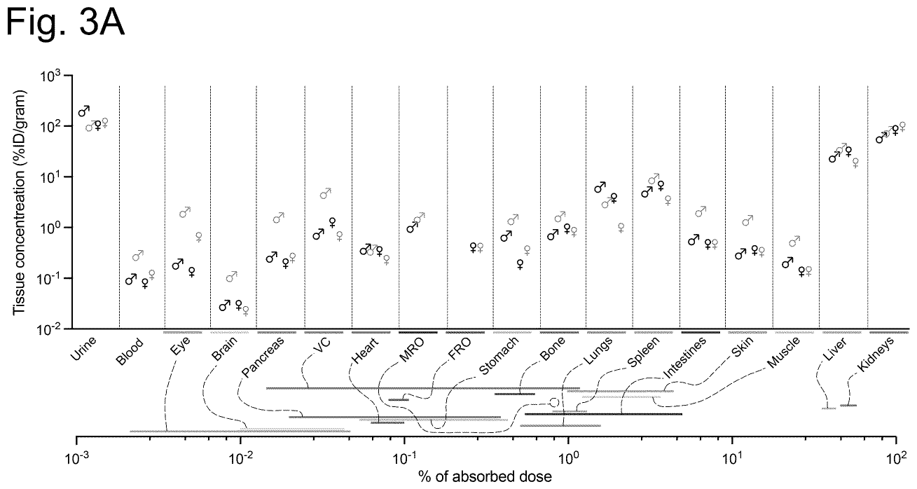

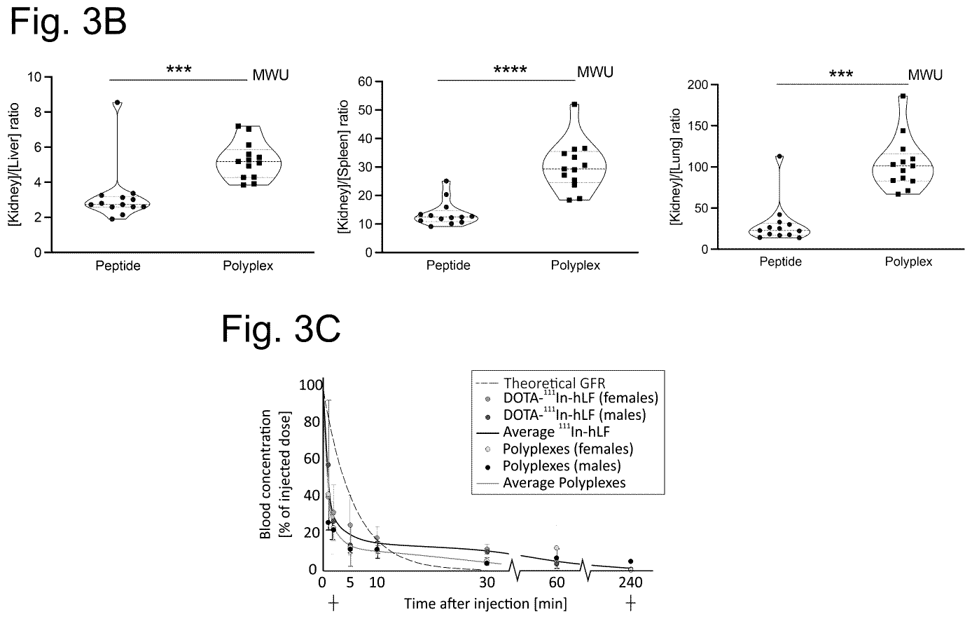

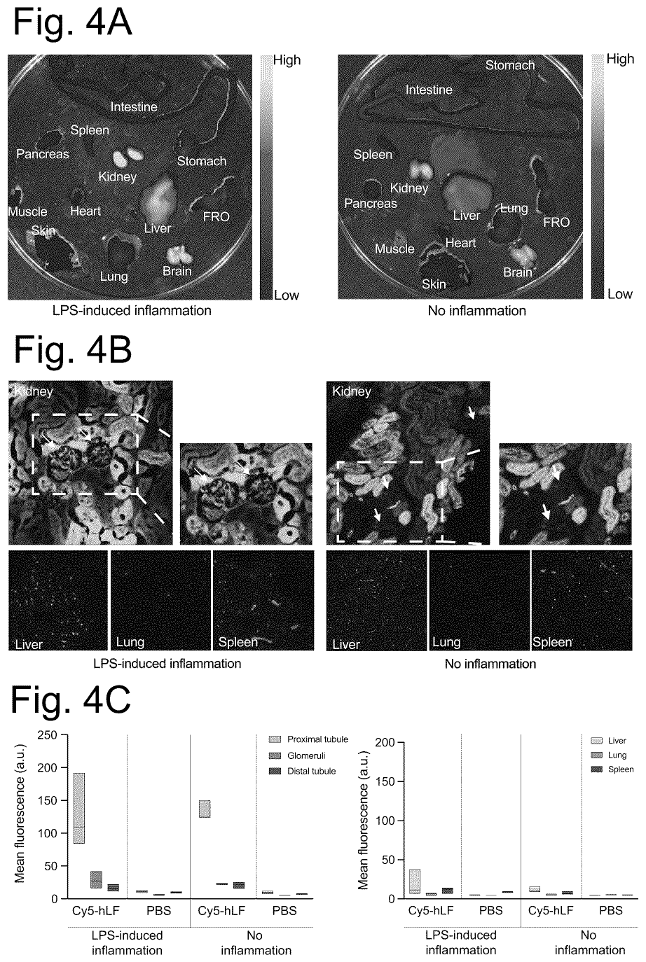

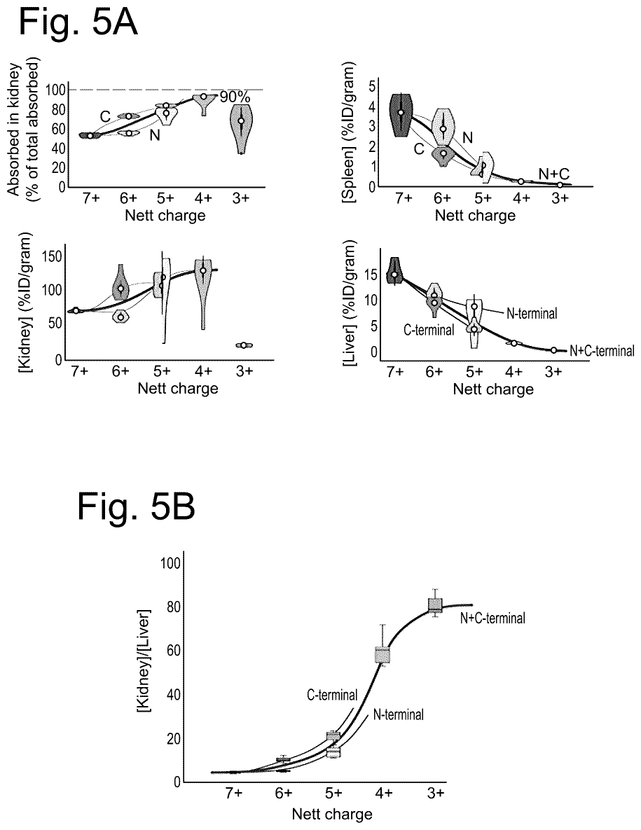

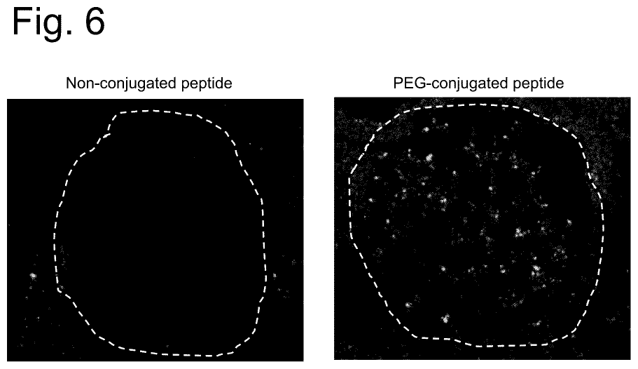

Incubation of multiple cell-penetrating peptides with mGEnC revealed that the hLF peptide showed a much more efficient dose-dependent uptake than two well-known cell-penetrating peptides, nona-arginine (R9) and penetratin (FIG. 1A). In contrast, in Hela cells all peptides exhibited an equal uptake efficiency at lower concentrations (FIG. 1B), while at 20 μM R9 showed a highly efficient uptake as observed before (Duchardt et al. J Biol Chem. 2009; 284 (52): 36099-108). The preferential uptake of hLF by mGEnC was not due to a difference in proteolytic breakdown of the peptides before uptake, or a difference in the binding to serum proteins. We also tested uptake of the mouse variant of hLF (designated as mLF), which contains a less positive net charge (Table 1). In mGEnC uptake occurred similarly to hLF (FIG. 1A). In contrast, Hela cells showed only a minimal uptake of mLF (FIG. 1B). Variants of the hLF peptide that lacked the disulphide bridge, or consisted out of D-amino acids, both showed a largely reduced uptake in mGEnC.

In order to further substantiate that uptake of hLF occurred by a mechanism different of the one typically observed for CPPs, we addressed the role of heparan sulphate proteoglycans (HSPGs). We previously demonstrated that in Hela cells the uptake of hLF, in accordance with prototypic CPPs, depends on the presence of HSPG (Duchardt et al. J Biol Chem. 2009; 284 (52): 36099-108; Wallbrecher et al. Cell Mol Life Sci. 2014; 71 (14): 2717-2729). For glomerular endothelial cells, the removal of heparan sulphate by treatment with heparinases only resulted in a minor decrease (~20%) in the uptake of hLF and mLF, while the uptake of R9 was reduced by approximately 60% (FIG. 1D).

Inside mGEnC, the lactoferrin-derived peptides showed a punctate staining, indicative of endocytic uptake (FIG. 1C). Fluorescence co-localized almost completely with fluorescently-labelled transferrin, a marker for clathrin-dependent endocytosis.

Interestingly, treatment of mGEnC with TNFα, which results in pro-inflammatory activation of the cells, augmented uptake of hLF and penetratin by 50-100%, however the uptake of penetratin was still barely detectable by confocal microscopy (FIG. 1E). In addition, the uptake of transferrin was not significantly affected by TNFα treatment, indicating no general effect on clathrin-mediated endocytosis.

### Interpretation

Here, we have shown that a lactoferrin-derived 22-amino acid peptide, previously identified as a CPP demonstrates uptake that largely exceeded that of other cell-penetrating peptides. While on Hela cells, uptake activity was similar to the prototypic CPPs nona-arginine and penetratin. The fact that the mLF peptide, which has only very little activity as a CPP on Hela cells, also exhibited strong uptake, suggests a receptor-dependent uptake mechanism. This was strengthened by the exclusive co-localization of the peptide with transferrin, providing evidence for clathrin-dependent endocytosis as the route of uptake, in contrast to prototypic CPPs that simultaneously engage different endocytic uptake routes (Duchardt et al. Traffic 2007; 8 (7): 848-866). Finally, the limited role of heparan sulphate in the uptake of lactoferrin-derived peptides by glomerular endothelial cells was contradictory to the heparan sulphate-dependent uptake of CPPs in Hela cells (Favretto et al. J Contr Rel. 2014; 180:81-90, Duchardt et al. J Biol Chem. 2009; 284 (52): 36099-108).

TABLE 1
Primary structure of the peptides that were used in this study.

| SEQ ID NO | | |
|---|---|---|
| of amino | | |
| acid | Peptide | |
| backbone | (net charge) | Sequence |
| 85 | Cy5.5-hLF | Cy5.5-KCFQWQRNMRKVRGPPVSCIKR-NH₂* |
| | (7+) | |
| 86 | Fluo-hLF (7+) | Fluo-KCFQWQRNMRKVRGPPVSCIKR-NH₂^{#}* |
| 87 | Fluo-mLF (5+) | Fluo-KCLRWQNEMRKVGGPPLSCIKR-NH₂^{#}* |
| 88 | Fluo-R9 | Fluo-RRRRRRRRR-NH₂^{#}* |
| | (9+) | |
| 89 | Fluo-penetratin | Fluo-RQIKIWFQNRRMKWKK-NH₂^{#}* |
| | (7+) | |
| 90 | ¹¹¹In-DOTA-hLF | ¹¹¹In-DOTA-KCFQWQRNMRKVRGPPVSCIKR-NH₂* |
| | (7+) | |
| 91 | ¹¹¹In-DOTA-hLF-3 | ¹¹¹In-DOTA-KCFQWQRNMRKVRGPPVSCIKRDS- |
| | (6+) | NH₂* |
| 92 | ¹¹¹In-DOTA-hLF-3 | ¹¹¹In-DOTA-CFQWQRNMRKVRGPPVSCIKR-NH₂* |
| | (6+) | |
| 93 | ¹¹¹In-DOTA-hLF-4 | ¹¹¹In-DOTA-KCFQWQRNMRKVRGPPVSC-NH₂* |
| | (5+) | |
| 94 | ¹¹¹In-DOTA-hLF-5 | ¹¹¹In-DOTA-RKVRGPPVSCIKR-NH₂* |
| | (5+) | |
| 95 | ¹¹¹In-DOTA-hLF-6 | ¹¹¹In-DOTA-RKVRGPPVSCIKRDS-NH₂* |
| | (4+) | |
| 96 | ¹¹¹In-DOTA-hLF-7 | ¹¹¹In-DOTA-RKVRGPP-NH₂* |
| | (3+) | |
| 97 | Cy5.5-hLF-6 | Cy5.5-RKVRGPPVSCIKRDS-NH₂* |
| | (4+) | |

*-NH2 indicates a C-terminal amide, ^{#}Fluo indicates an N-terminal carboxyfluorescein

### Example 2

This example illustrates the presence of ITLN-1 on glomerular endothelial cells and its involvement in the uptake of the hLF peptide.

### Methods

Conditionally immortalized mouse glomerular endothelial cells (mGEnC) were cultured as previously described (Rops et al. Kidney Int. 2004; 66 (6): 2193-201). Cells were incubated in their respective medium with 10% FCS containing fluorescein-labelled peptides at 5 μM for 20 minutes at 37 °C. In some cases, cells were activated for 16 hours with 10 ng/ml mouse TNFα (Sigma), or co-incubated with 100 μg/ml Alexa Fluor 633-labeled transferrin (Invitrogen Molecular Probes). Pre-treatment with ITLN-1 or control siPool siRNA (siTools Biotech) was performed according to the manufacturer's protocol. Subsequently, cells were detached with trypsin/EDTA and analysed by flow cytometry on a FACSCalibur Flow cytometer (BD Biosciences). For immunostaining, cells or 5-μm cryosections of mouse kidney were fixed for 10 minutes with 4% paraformaldehyde, and in the indicated cases permeabilized with 0.3% Triton X-100 in PBS. Subsequently, cells and cryosections were stained for 1 hour with anti-intelectin-1 (R&D Systems) or anti-LRP-1 (Abcam) antibodies, and after washing incubated for 1 hour with the appropriate Alexa Fluor-labelled secondary antibody (Invitrogen Molecular Probes). Cells and cryosections were analyzed by confocal laser-Scanning microscopy was performed using a TCS SP5 confocal microscope (Leica Microsystems).

### Results

Glomerular endothelial cells were tested for the expression of two previously described lactoferrin-receptors, i.e. ITLN-1 and low density lipoprotein receptor related protein 1 (LRP-1) (Suzuki et al. Biochemistry 2001; 40 (51): 15771-15779; Willnow et al. J|Biol Chem. 1992; 267 (36): 26172-26180). We found that ILTN-1 was expressed on the membrane of these cells (FIG. 2A), and was strongly present in the cytoplasm of these cells (FIG. 2A). In contrast, we did not observe significant expression of LRP-1 by flow cytometry or confocal laser-scanning microscopy. Interestingly, ITLN-1 expression coincided with the uptake of the non-CPP mLF in multiple endothelial cell lines, including ciGEnC, HUVEC and EOMA, while HeLa cells lacked expression of ITLN-1 (Table 2). Moreover, activation of mGEnC by TNFα augmented ITLN-1 expression, in line with the increased uptake of hLF and mLF after activation (FIG. 2B). We were unable to directly visualize co-localization of ITLN-1 with fluorescein-labelled hLF after uptake, as the peptide could not be fixated. Therefore, we co-incubated mGEnC with hLF and fluorescently-labelled transferrin, which co-localized completely with hLF (FIG. 2C), and stained for ITLN-1. This revealed a prominent intracellular co-localization of ITLN-1 with transferrin-containing vesicles (FIG. 2D). To directly demonstrate the involvement of ITLN-1 in the uptake of lactoferrin-derived peptides, we pre-treated mGEnC with siRNA for ITLN-1, resulting in an almost complete down regulation of the protein (FIG. 2E, right top panel). Cells treated with ITLN-1 siRNA exhibited a reduced uptake for hLF and mLF, compared to treatment with control siRNA, while the uptake of R9, penetratin and transferrin was not affected (FIG. 2E, bar graph panel). Finally, we stained kidney cryosections of BALB/c mice and MRL/MpJ mice (healthy (middle panel) and diseased (right panel)) for ITLN-1. Expression of ILTN-1 protein could be observed in the glomeruli and peritubular capillaries (FIG. 2F).

### Interpretation

The uptake characteristics of hLF in glomerular endothelial cells strongly suggested that uptake of the lactoferrin-derived peptides might involve a specific receptor. We confirmed the involvement of ITLN-1 in the uptake of lactoferrin-derived peptides, since downregulation by ITLN-1 siRNA decreased uptake of the lactoferrin-derived peptides, but not that of prototypic CPPs. We could show that glomerular endothelial cells express ITLN-1, while this receptor was not expressed in cells that do not show the specific uptake of hLF. In contrast, we found no expression of another described receptor for hLF, LRP-1. In addition, we could demonstrate the presence of ITLN-1 in glomeruli of normal BALB/c and MRL/MpJ mice (background strain), and MRL/MpJ-FasIpr mice that have developed lupus-associated glomerular inflammation. Altogether, these data indicate that ITLN-1 contributes to the uptake of lactoferrin-derived peptides in glomerular endothelial cells.

TABLE 2
The uptake of lactoferrin-derived CPP, and the expression
of the lactoferrin receptor in ITLN-1 in multiple
endothelial and epithelial cell lines.

| | | | Uptake | |
|---|---|---|---|---|
| | Uptake | Uptake | of R9 and | Expression |
| Cell line | of hLF¹ | of mLF¹ | penentratin¹ | of ITLN-1² |
| mGEnC (mouse) | High | High | Low | High |
| hGEnC (human) | High | High | Low | High |
| EOMA (mouse) | High | High | Low | ND³ |
| HUVEC (human) | High | High | High | High |
| Podocytes (mouse) | High | High | High | High |
| HeLa (human) | High | Low | High | Low |

¹As determined by the uptake of a fluorescein-labeled peptide using confocal microscopy
²As determined by confocal microscopy after staining with an anti-ITLN-1 antibody, in which “low” represents a barely detectable signal and “high” a prominent staining
³ND, no data

### Example 3

This example illustrates that after intravenous injection the hLF peptide predominantly distributes to the kidneys of normal, healthy mice.

### Methods

Female and male 10-12-weeks-old C57BI/6 mice (Charles River) were injected via the tail vein with 50 μl of 200 μM 111 In-DOTA-labelled hLF peptide. Assuming a blood volume of 1.2 ml this would theoretically result in a concentration of ~8.5 μM in the circulation. Urine, blood and organs were collected 4 hours after injection, and radioactivity was measured in a gamma-counter. The percentage injected dose per gram of tissue (% ID/gram) was calculated for each tissue along with % of the absorbed dose.

### Results

Biodistribution of the hLF peptide was predominantly located in the kidney of both female and male mice (FIG. 3A). Distribution to the liver was approximately 2-fold lower, while spleen and lungs contained a 5-10-fold lower peptide concentration. Other organs, including intestines, skin and heart, showed more than 20-fold lower distribution of the hLF peptide. The percentage of the absorbed dose in the kidney and liver combined comprised more than 90% of the total dose in all the organs. After 4 hours, the concentration in the blood was <1% and the hLF peptide was either taken up by cells or excreted in the urine.

### Interpretation

The surprising predominant distribution of the hLF peptide to the kidney suggests a specific uptake mechanism in renal cells. Based on the fact that the hLF peptide has activity as a CPP, a rapid uptake in most well-perfused organs was expected as was shown previously for a selection of different CPPs (Sarko et al. Mol Pharmaceut. 2010; 7 (6): 2224-2231). In addition, the biodistribution of radioactive-labelled full-length human lactoferrin protein was previously shown to comprise multiple organs and not primarily the kidney (Kanoun et al. J Radioanalytical Nuclear Chem. 2018; 317:177-185). Usage of the full-length lactoferrin protein for targeting nanoparticles resulted in enhanced uptake in the liver, spleen and brain, while uptake in the kidney was decreased (Qi et al. J Nanobiotechnology. 2021; 19:446; Kaili et al. Int J Pharmaceutics. 2011; 415:273-283; Farhan et al. Environmental Sci Pollution Res. 2018; 9:1-17). In contrast, in our case, organs for which other potential receptors of full-length lactoferrin have been described such as the liver, spleen, lungs and intestines, absorbed less than 5% of the lactoferrin-derived peptide. The predominance of the enrichment in the kidneys corroborates the ITLN-1-mediated uptake that we identified in glomerular cells. This distribution also suggests that the uptake of hLF via ITLN-1 exceeds the uptake via the mechanisms associated with prototypic CPPs and thus that hLF acts by targeting the ITLN-1 and not as a CPP as described in the prior art. Furthermore, the found bio-distribution pattern is also an indicator of the absence of binding to other receptors, proteins, sugars, and/or cellular components that would result in uptake in other organs. No significant differences were observed for the kidney targeting between female and male mice.

### Example 4

This example illustrates that complexation of the hLF peptide with mRNA results in an enhanced distribution to the kidney.

### Methods

Polyplexes of hLF peptide and mRNA were formed by fast-fluid mixing using an N/P ration of 3. Complexes were subsequently measured for their size using dynamic light scattering (DLS). Female and male 10-12-weeks-old C57BI/6 mice (Charles River) were injected via the tail vein with 50 μl of 200 μM ¹¹¹In-DOTA-labelled hLF peptide loose or complexed with mRNA. Assuming a blood volume of 1.2 ml this would theoretically result in a concentration of ~8.5 M in the circulation. Urine, blood and organs were collected 4 hours after injection, and radioactivity was measured in a gamma-counter. The percentage injected dose per gram of tissue (% ID/gram) was calculated for each tissue. For determination of the peptide blood concentration, blood was drawn from mice at several timepoints (1-240 minutes).

### Results

Intravenous injection of polyplexes of hLF/mRNA, which were ~80 nm in size, resulted in a similar peptide concentration in the kidneys compared to free peptide (FIG. 3A). Surprisingly, we observed a decrease in the distribution to the liver, spleen and lungs (FIG. 3B). The kidney/liver ratio for polyplexes was increased ~2.5-fold compared to free peptide, while the kidney/spleen ration (3-fold) and kidney/lung ratio (6-fold) showed an even larger significant increase. Differences in other organs had only a marginal impact due to the low amount of peptide concentration in the respective organ. Additionally, we observed a rapid blood clearance of the peptide, either free or complexed with mRNA, which is faster than the theoretical glomerular filtration rate (GFR) (FIG. 3C).

### Interpretation

The relative increase in renal biodistribution after complexation of hLF suggest a role of multi-valency in the uptake mechanism. The presence of multiple hLF peptides, and thus multiple available ITLN-1 binding epitopes, on the surface of polyplexes would favour receptor-mediated uptake. The reduction in uptake in other organs, including the liver, spleen and lungs, might be explained by their dependency on positive charged residues, which would be partially covered by the mRNA. The rapid blood clearance is in line with active peptide binding and/or uptake.

### Example 5

This example illustrates the enhanced biodistribution of intravenous-injected hLF peptide to glomerular cells in the kidneys of mice with LPS-induced renal inflammation.

### Methods

At the start, 8- to 10-week-old C57BI/6 mice (Charles River) received an intraperitoneal injection with either 100 μl of 2 mg/kg LPS in 5% glucose solution, or only 5% glucose solution. After 24 hours, mice were injected with 50 μl of 200 μM Cy5.5-labelled peptide. Assuming a blood volume of 1.2 ml this would theoretically result in a concentration of ~8.5 μM in the circulation. After 4 hours, organs were perfused with phosphate buffer, followed by 4% PFA. Organs were removed and peptide distribution was analysed using the In Vivo Imaging System (IVIS; Perkin Elmer). Organs were then fixed by overnight incubation in 4% PFA, followed by overnight incubation in 30% sucrose. Subsequently, organs were frozen for analysis by confocal laser-Scanning microscopy using a TCS SP5 confocal microscope (Leica Microsystems). Glomerular and tubular regions were selected in microscopical pictures from which the mean fluorescence intensity was determined by ImageJ software.

### Results

Analysis of removed organs by IVIS revealed an increase biodistribution to the kidney in mice with LPS-induced inflammation (FIG. 4A). For both LPS-treated and non-treated mice, the highest signal for the peptide was present in the kidneys, followed by the liver, while the hLF peptide was virtually undetectable in spleen, heart, eyes, brain and lymph nodes. Urine was coloured by excreted Cy5.5-labelled peptide in all mice, except for the non-injected mouse. Histological analysis of cryosections revealed that localization in the glomeruli was clearly pronounced in kidney of LPS-treated mice, while localization to the tubules was similar (FIG. 4B). In addition, histological analysis confirmed that Cy5.5-labeled hLF could hardly be detected in the spleen, while singular cells appeared weakly positive in the liver. Software analysis of multiple pictures of renal cryosections confirmed the increased in glomerular localization (FIG. 4C).

### Interpretation

Induction of renal inflammation by LPS, a well-established model for acute renal inflammation, results in an increase in biodistribution of Cy5.5-labelled hLF peptide to the glomeruli. The glomerular targeting is in line with the ITLN-1-dependent uptake observed in vitro in glomerular cell lines, the inflammation-induced increase in receptor expression, and the exclusive glomerular localization of ITLN-1 in the kidney. Importantly, this means that pathophysiological relevant glomerular cell types, which are associated with CKD, are reached. Tubular uptake of the peptide is likely due endocytic clearance via megalin or cubulin receptors that are involved in the reabsorption of a wide array of proteins.

### Example 6

This example illustrates the development of hLF peptide derivatives that demonstrate an enhanced targeting to the kidney.

### Methods

Female and male 10-12-weeks-old C57BI/6 mice (Charles River) were injected via the tail vein with 50 μl of 200 μM 111 In-DOTA-labelled hLF peptides. The hLF peptide variants possessed different overall charges where charges were reduced either on the N-terminal and/or C-terminal part (Table 1, SEQ ID NO 90-97). Organs were collected 4 hours after injection, and radioactivity was measured in a gamma-counter. The percentage injected dose per gram of tissue (% ID/gram) was determined for each tissue and ratios between organs were calculated.

### Results

Intravenous injection of radioactive-labelled hLF variants with alterations to the N- and/or C-terminal side that resulted in a decreased net charge demonstrated an increased localization to the kidney, while localization to the liver and spleen was decreased (FIG. 5). This resulted in absorption in the kidney of up to 90% of the total amount of absorbed peptide in the mouse. Indeed, the kidney/liver ratio increased more than 30-fold from approximately 2.5 to 80. Exception was the shortest hLF peptide variant that was comprised of 7 amino acids and possessed a net charge 3+ (see Table 1, SEQ ID NO 96), which showed a reduced distribution to the kidney along with a reduced distribution to the liver and spleen. However, kidney/liver ratio was still increased to peptides with a lower net charge.

### Interpretation

Altering the net charge of the hLF peptide by addition or removal of naturally-occurring amino acids resulted in a large improvement of its kidney targeting efficacy. Previous studies showed that the kidney and liver targeting of CPPs is not per se dependent on the net charge of the CPP (Sarko et al. Mol Pharmaceut. 2010; 7 (6): 2224-2231). Therefore, it was surprising that reducing net charge of the hLF peptide reduced uptake in the liver, spleen and lungs, but not in the kidney. In addition, shortening the peptide, which might affect binding to the receptor, resulted in an increased uptake in the kidney. Further reduction of the length to 7 amino acids did reduce off-targeting to other organs, but also negatively affected uptake in the kidney, leading to an increased excretion of the peptide in the urine.

### Example 7

This example illustrates that conjugation of an improved hLF variant to a larger molecule that cannot cross the kidney filter results in exclusive renal distribution to the glomeruli, while tubular uptake is evaded.

### Methods

Cy5.5-labelled hLF-6 (Table 1) was dissolved in 4 mM citrate buffer pH 5, to prevent reactivity of cysteines. Conjugation was performed for 2 hours in the dark in 40 mM phosphate buffer pH 7 by adding 40-kDa-PEG-maleimide to achieve a PEG:peptide ratio of 1:10. At the start, 8- to 10-week-old C57BI/6 mice (Charles River) received an intraperitoneal injection with either 100 μl of 2 mg/kg LPS in 5% glucose solution, or only 5% glucose solution. After 24 hours, mice were injected 50 μl of 200 μM Cy5.5-labelled PEG-hLF-6. After 4 hours, organs were perfused with phosphate buffer, followed by 4% PFA. Organs were removed and peptide distribution was analysed using the In Vivo Imaging System (IVIS; Perkin Elmer). Organs were further fixed by overnight incubation in 4% PFA, followed by overnight incubation in 30% sucrose. Subsequently, organs were frozen for analysis by confocal laser-Scanning microscopy using a TCS SP5 confocal microscope (Leica Microsystems).

### Results

Analysis of renal cryosection by confocal microscopy revealed that PEG-hLF-6 was exclusively localized in glomeruli, while we did not observe localization in proximal tubules (FIG. 6). The PEG-hLF-6 was present as multiple larger and smaller dots throughout the inside of the glomerulus. Urine that was obtained from these mice was not (fluorescently) coloured.

### Interpretation

Conjugation of an improved variant of hLF to 40-kDa PEG results in an altered biodistribution in the kidney after intravenous injection into mice. Where we observed strong tubular localization when injected free peptide in LPS-induced mice, along with a weaker staining in the glomerulus, conjugation results in an exclusive localization in the glomerulus. This suggests that attaching the peptide to a nanoparticle that cannot pass the glomerular filter (>30-50 nm), prevents leakage to the tubular compartment and uptake in tubular cells via scavenger receptors such as megalin and cubulin. This is also corroborated by the fact that we could not observe excretion of peptide into the urine.

### Example 8

This example illustrates that full-length lactoferrin protein and its natural derivate lactoferricin do not have the same (kidney-specific) bio-distribution as the lactoferrin-derived peptides of the invention.

### Methods

Lactoferrin and lactoferricin are cloned in an expression plasmid containing the C-terminal Sortase-sequence protein-LPETG-HHHHHH. LPETGHHHHHH is SEQ ID NO: 98. Next, the protein is expressed overnight in E. coli and purified from sonicated bacterial cell lysates by Ni-NTA beads using 400 mM imidazole to elute the proteins. After overnight dialysis, full-length lactoferrin and hLF-6 are labelled by means of a Sortase exchange reaction on the C-terminus with a fluorescent dye (Alexa647 or Cy5.5) for 2 h at 4 °C using a fluorescently labelled donor peptide (formula:biotin-GSSG-LPETG-label or biotin-GSSG-LPETG-PEG40k-label) as substrate GSSGLPETG is SEQ ID NO: 99. Excess donor peptide is removed by means of streptavidin binding of the biotin tag on the N-terminus of the donor peptide. Next, the purified labelled protein/peptide is injected into LPS-challenged mice via intravenous injection in the tail vein. Mice are sacrificed after 2 h, and organs harvested for imaging by IVIS. Biodistribution is quantified for each organ, and relative distribution is shown.

### Results

Bio-distribution imaging of full-length lactoferrin and lactoferricin shows accumulation predominantly in liver and spleen and less in kidney, brain and intestine. In contrast, the hLF-6-peptide, with and without the 40 kDa PEG linker, shows significantly greater accumulation in the kidney.

### Interpretation

The distinct bio-distribution of hLF-6 (see Table 1), as an example of the peptides in this invention, from full-length lactoferrin and lactoferricin demonstrates that removing binding domains to other receptors leads to a more specific targeting. This also differentiates the present approach from the prior art, where biodistribution to multiple organs and a very limited biodistribution to specific cell-types was also revealed. The differences in biodistribution are most likely due to differences in co-receptor binding and/or other factors, be they known or not.

1. A complex comprising a cargo and a targeting moiety binding intelectin-1 (ITLN-1),
wherein the targeting moiety is a peptide,
wherein the peptide has a length of at least 6 and at most 30 amino acids, and
wherein the peptide comprises or consists of a peptide selected from the group consisting of SEQ ID NOs: 3-84.

2. The complex for use-according to claim 1, wherein the peptide has a length of at least 7 and at most 24 amino acids.

3. The complex for use-according to claim 1, wherein the peptide comprises or consists of a consecutive sequence of at least 6 amino acids selected from the sequences represented by any one of SEQ ID NOs: 80-84.

4. The complex according to claim 1, wherein affinity of the complex to ITLN-1 is enhanced by the multi-valent use of the targeting moiety.

5. The complex according to claim 14, wherein the complex is suitable for use as an agent to bind to, to target to, to purify, to induce uptake into cells or to transfect cells expressing ITLN-1 comprising administration of the complex to a subject.

6. The complex according to claim 1, wherein the cargo is a nucleic acid, a peptide, polypeptide or protein, a carbohydrate, a lipid, a polymer, a small molecule, or a mixture thereof.

7. The complex according to claim 1, wherein the cargo is a vehicle such as a particle, such as a micro-particle or a nano-particle, a liposome, a lipid nanoparticle, a polymer particle, a silica particle, a carbon-nanotube, a gold particle, or a lipid- or polymer-micelle.

8. The complex according to claim 5, wherein the complex is for targeting a cell, wherein the cell is an ITLN1-expressing kidney cell, such as a glomerular endothelial cell or a podocyte.

9. The complex according to claim 5, wherein administration of the complex to a subject results in biodistribution of the complex predominantly to ITLN1-expressing cells, such that the cargo accumulates at least 1.5-fold, at least 2-fold, at least 3-fold, at least 4-fold, at least 5-fold, at least 6-fold, at least 7-fold, at least 8-fold, at least 9-fold, preferably- or at least 10-fold more in the organ containing ITLN1-expressing cells than in the off-target organs, wherein off-target organ refers to an organ not containing ITLN1-expressing cells.

10.-11. (canceled)

12. Method for treating a kidney, eye, lung, or intestinal disease, the method comprising the step of administering the complex according to claim 1 to a subject.

13. The method according to claim 12, wherein the disease is kidney disease.

14. The method according to claim 12, wherein the cargo is an anti-inflammatory drug, an anti-fibrotic drug, a steroid, a protein kinase inhibitor, a gene-correcting agent, an oligonucleotide, or wherein the cargo is messenger RNA, circular RNA, trans-amplifying RNA, self-amplifying RNA, or DNA.

15. The method according to claim 12, wherein the cargo is an anti-inflammatory drug or an anti-fibrotic drug.

16. The method according to claim 12, for use in the treatment of a condition associated with upregulation, over-expression and/or increased availability of ITLN-1.

17. The method according to claim 16, wherein the condition is inflammation such as kidney inflammation and such as ulcerative colitis, or wherein the condition is cancer, such as prostate cancer.

18. The method according to claim 12, wherein clearance of the cargo from the system is accelerated compared to the cargo without targeting moiety.

19. The method according to claim 18, wherein clearance from the system is clearance from the blood circulatory system.

20. (canceled)

21. A method to bind, target, purify, induce uptake, or transfect cells expressing ITLN-1, the method comprising contacting a complex as defined in claim 1 with the cells.

22. The method according to claim 21, wherein the method is in vitro or ex vivo.

23. The method according to claim 21, wherein the uptake is endosomal uptake, or wherein the contacting is under conditions that allow for receptor-specific binding and/or uptake.

24.-36. (canceled)


SEQUENCE LISTING (99 sequences)

SEQ ID NO: 1 - protein, 710 aa, synthetic construct
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 2 - protein, 708 aa, synthetic construct
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 3 - protein, 22 aa, synthetic construct
KCFQWQRNMRKVRGPPVSCIKR

SEQ ID NO: 4 - protein, 22 aa, synthetic construct
KCLRWQNEMRKVGGPPLSCIKR

SEQ ID NO: 5 - protein, 24 aa, synthetic construct
KCFQWQRNMRKVRGPPVSCIKRDS

SEQ ID NO: 6 - protein, 21 aa, synthetic construct
CFQWQRNMRKVRGPPVSCIKR

SEQ ID NO: 7 - protein, 19 aa, synthetic construct
KCFQWQRNMRKVRGPPVSC

SEQ ID NO: 8 - protein, 13 aa, synthetic construct
RKVRGPPVSCIKR

SEQ ID NO: 9 - protein, 15 aa, synthetic construct
RKVRGPPVSCIKRDS

SEQ ID NO: 10 - protein, 7 aa, synthetic construct
RKVRGPP

SEQ ID NO: 11 - protein, 23 aa, synthetic construct
KCFQWQRNMRKVRGPPVSCIKRD

SEQ ID NO: 12 - protein, 21 aa, synthetic construct
KCFQWQRNMRKVRGPPVSCIK

SEQ ID NO: 13 - protein, 20 aa, synthetic construct
KCFQWQRNMRKVRGPPVSCI

SEQ ID NO: 14 - protein, 19 aa, synthetic construct
KCFQWQRNMRKVRGPPVSC

SEQ ID NO: 15 - protein, 18 aa, synthetic construct
KCFQWQRNMRKVRGPPVS

SEQ ID NO: 16 - protein, 17 aa, synthetic construct
KCFQWQRNMRKVRGPPV

SEQ ID NO: 17 - protein, 16 aa, synthetic construct
KCFQWQRNMRKVRGPP

SEQ ID NO: 18 - protein, 21 aa, synthetic construct
CFQWQRNMRKVRGPPVSCIKR

SEQ ID NO: 19 - protein, 20 aa, synthetic construct
FQWQRNMRKVRGPPVSCIKR

SEQ ID NO: 20 - protein, 19 aa, synthetic construct
QWQRNMRKVRGPPVSCIKR

SEQ ID NO: 21 - protein, 18 aa, synthetic construct
WQRNMRKVRGPPVSCIKR

SEQ ID NO: 22 - protein, 17 aa, synthetic construct
QRNMRKVRGPPVSCIKR

SEQ ID NO: 23 - protein, 16 aa, synthetic construct
RNMRKVRGPPVSCIKR

SEQ ID NO: 24 - protein, 15 aa, synthetic construct
NMRKVRGPPVSCIKR

SEQ ID NO: 25 - protein, 14 aa, synthetic construct
MRKVRGPPVSCIKR

SEQ ID NO: 26 - protein, 13 aa, synthetic construct
RKVRGPPVSCIKR

SEQ ID NO: 27 - protein, 22 aa, unidentified
CFQWQRNMRKVRGPPVSCIKRD

SEQ ID NO: 28 - protein, 21 aa, synthetic construct
FQWQRNMRKVRGPPVSCIKRD

SEQ ID NO: 29 - protein, 20 aa, synthetic construct
QWQRNMRKVRGPPVSCIKRD

SEQ ID NO: 30 - protein, 19 aa, synthetic construct
WQRNMRKVRGPPVSCIKRD

SEQ ID NO: 31 - protein, 18 aa, synthetic construct
QRNMRKVRGPPVSCIKRD

SEQ ID NO: 32 - protein, 17 aa, synthetic construct
RNMRKVRGPPVSCIKRD

SEQ ID NO: 33 - protein, 16 aa, synthetic construct
NMRKVRGPPVSCIKRD

SEQ ID NO: 34 - protein, 15 aa, synthetic construct
MRKVRGPPVSCIKRD

SEQ ID NO: 35 - protein, 14 aa, synthetic construct
RKVRGPPVSCIKRD

SEQ ID NO: 36 - protein, 22 aa, synthetic construct
FQWQRNMRKVRGPPVSCIKRDS

SEQ ID NO: 37 - protein, 22 aa, synthetic construct
FQWQRNMRKVRGPPVSCIKRDS

SEQ ID NO: 38 - protein, 21 aa, synthetic construct
QWQRNMRKVRGPPVSCIKRDS

SEQ ID NO: 39 - protein, 20 aa, synthetic construct
WQRNMRKVRGPPVSCIKRDS

SEQ ID NO: 40 - protein, 19 aa, synthetic construct
QRNMRKVRGPPVSCIKRDS

SEQ ID NO: 41 - protein, 18 aa, synthetic construct
RNMRKVRGPPVSCIKRDS

SEQ ID NO: 42 - protein, 17 aa, synthetic construct
NMRKVRGPPVSCIKRDS

SEQ ID NO: 43 - protein, 16 aa, synthetic construct
MRKVRGPPVSCIKRDS

SEQ ID NO: 44 - protein, 15 aa, synthetic construct
RKVRGPPVSCIKRDS

SEQ ID NO: 45 - protein, 18 aa, synthetic construct
CFQWQRNMRKVRGPPVSC

SEQ ID NO: 46 - protein, 17 aa, synthetic construct
FQWQRNMRKVRGPPVSC

SEQ ID NO: 47 - protein, 16 aa, synthetic construct
QWQRNMRKVRGPPVSC

SEQ ID NO: 48 - protein, 15 aa, synthetic construct
WQRNMRKVRGPPVSC

SEQ ID NO: 49 - protein, 14 aa, synthetic construct
QRNMRKVRGPPVSC

SEQ ID NO: 50 - protein, 13 aa, synthetic construct
RNMRKVRGPPVSC

SEQ ID NO: 51 - protein, 12 aa, synthetic construct
NMRKVRGPPVSC

SEQ ID NO: 52 - protein, 11 aa, synthetic construct
MRKVRGPPVSC

SEQ ID NO: 53 - protein, 10 aa, synthetic construct
RKVRGPPVSC

SEQ ID NO: 54 - protein, 17 aa, synthetic construct
CFQWQRNMRKVRGPPVS

SEQ ID NO: 55 - protein, 16 aa, synthetic construct
FQWQRNMRKVRGPPVS

SEQ ID NO: 56 - protein, 15 aa, unidentified
QWQRNMRKVRGPPVS

SEQ ID NO: 57 - protein, 14 aa, synthetic construct
WQRNMRKVRGPPVS

SEQ ID NO: 58 - protein, 13 aa, synthetic construct
QRNMRKVRGPPVS

SEQ ID NO: 59 - protein, 12 aa, synthetic construct
RNMRKVRGPPVS

SEQ ID NO: 60 - protein, 11 aa, synthetic construct
NMRKVRGPPVS

SEQ ID NO: 61 - protein, 10 aa, synthetic construct
MRKVRGPPVS

SEQ ID NO: 62 - protein, 9 aa, synthetic construct
RKVRGPPVS

SEQ ID NO: 63 - protein, 16 aa, synthetic construct
CFQWQRNMRKVRGPPV

SEQ ID NO: 64 - protein, 15 aa, synthetic construct
FQWQRNMRKVRGPPV

SEQ ID NO: 65 - protein, 14 aa, synthetic construct
QWQRNMRKVRGPPV

SEQ ID NO: 66 - protein, 13 aa, synthetic construct
WQRNMRKVRGPPV

SEQ ID NO: 67 - protein, 12 aa, synthetic construct
QRNMRKVRGPPV

SEQ ID NO: 68 - protein, 11 aa, synthetic construct
RNMRKVRGPPV

SEQ ID NO: 69 - protein, 10 aa, synthetic construct
NMRKVRGPPV

SEQ ID NO: 70 - protein, 9 aa, synthetic construct
MRKVRGPPV

SEQ ID NO: 71 - protein, 8 aa, synthetic construct
RKVRGPPV

SEQ ID NO: 72 - protein, 15 aa, synthetic construct
CFQWQRNMRKVRGPP

SEQ ID NO: 73 - protein, 14 aa, synthetic construct
FQWQRNMRKVRGPP

SEQ ID NO: 74 - protein, 13 aa, synthetic construct
QWQRNMRKVRGPP

SEQ ID NO: 75 - protein, 12 aa, synthetic construct
WQRNMRKVRGPP

SEQ ID NO: 76 - protein, 11 aa, synthetic construct
QRNMRKVRGPP

SEQ ID NO: 77 - protein, 10 aa, synthetic construct
RNMRKVRGPP

SEQ ID NO: 78 - protein, 9 aa, synthetic construct
NMRKVRGPP

SEQ ID NO: 79 - protein, 8 aa, synthetic construct
MRKVRGPP

SEQ ID NO: 80-84 - skipped (5 entries)

SEQ ID NO: 85 - protein, 22 aa, synthetic construct
KCFQWQRNMRKVRGPPVSCIKR

SEQ ID NO: 86 - protein, 22 aa, synthetic construct
KCFQWQRNMRKVRGPPVSCIKR

SEQ ID NO: 87 - protein, 22 aa, synthetic construct
KCLRWQNEMRKVGGPPLSCIKR

SEQ ID NO: 88 - protein, 9 aa, synthetic construct
RRRRRRRRR

SEQ ID NO: 89 - protein, 16 aa, synthetic construct
RQIKIWFQNRRMKWKK

SEQ ID NO: 90 - protein, 22 aa, synthetic construct
KCFQWQRNMRKVRGPPVSCIKR

SEQ ID NO: 91 - protein, 24 aa, synthetic construct
KCFQWQRNMRKVRGPPVSCIKRDS

SEQ ID NO: 92 - protein, 21 aa, synthetic construct
CFQWQRNMRKVRGPPVSCIKR

SEQ ID NO: 93 - protein, 19 aa, synthetic construct
KCFQWQRNMRKVRGPPVSC

SEQ ID NO: 94 - protein, 13 aa, synthetic construct
RKVRGPPVSCIKR

SEQ ID NO: 95 - protein, 15 aa, synthetic construct
RKVRGPPVSCIKRDS

SEQ ID NO: 96 - protein, 7 aa, unidentified
RKVRGPP

SEQ ID NO: 97 - protein, 15 aa, synthetic construct
RKVRGPPVSCIKRDS

SEQ ID NO: 98 - protein, 11 aa, synthetic construct
LPETGHHHHHH

SEQ ID NO: 99 - protein, 9 aa, synthetic construct
GSSGLPETG